(19) United States
(12) Patent Application Publication
(10) Pub. No.: US 2005/0027300 A1
(43) Pub. Date: Feb. 3, 2005

(54) Method and apparatus for artificial disc insertion

(75) Inventors: John Riley Hawkins, Cumberland, RI (US); Shawn D. Stad, Fall River, MA (US); Christopher Rogers, Taunton, MA (US); Alexander Grinberg, Newton, MA (US); Ronald Naughton, Tiverton, RI (US); Michael D. Sorrenti, Middleboro, MA (US); Niall P. Casey, Boston, MA (US); Mark Gracia, Rochester, MA (US); Carl Souza, Dighton, MA (US); Pat Fatyol, Whitman, MA (US)

Correspondence Address: HAMILTON, BROOK, SMITH & REYNOLDS, P.C., 530 VIRGINIA ROAD, P.O. BOX 9133, CONCORD, MA, 01742-9133, US

(73) Assignee: DePuy Spine, Inc., Raynham, MA (US)

(21) Appl. No.: 10/813,899

(22) Filed: Mar. 31, 2004

Related U.S. Application Data
(60) Provisional application No. 60/459,280, filed on Mar. 31, 2003.

(51) Int. Cl. A61F 5/00

(52) U.S. Cl. 606/86

(57) ABSTRACT

An anterior method for implanting an artificial disc in an intervertebral space of a human body includes inserting a midline marker in a face of a vertebral body for instrument alignment and artificial disc placement. A kit for implanting an artificial disc in an intervertebral space of a human body includes site preparation instruments, artificial disc insertion instruments, and a midline marker for guiding the artificial disc insertion instruments into a prepared intervertebral space. Also included are a verification instrument, a midline marker, a midline marker insertion instrument, an endplate shaping device, a distraction instrument, a trial insertion instrument, an endplate insertion instrument, a core insertion instrument, and a trial spacer head.

### RELATED APPLICATIONS

This application claims the benefit of U.S. Provisional Application No. 60/459,280, filed Mar. 31, 2003. This application is related to U.S. patent application No. 10/011,264, filed Dec. 7, 2001; U.S. patent application No. 10/200,890, filed Jul. 23, 2002, U.S. Provisional Application No. 60/391,628, filed Jun. 26, 2002; and U.S. Provisional Application No. 60/391,845, filed Jun. 27, 2002. The entire teachings of the above applications are incorporated herein by reference.

### BACKGROUND OF THE INVENTION

An intervertebral disc has several important functions, including functioning as a spacer, a shock absorber, and a motion unit.

The disc maintains the separation distance between adjacent boney vertebral bodies. The separation distance allows motion to occur, with the cumulative effect of each spinal segment yielding the total range of motion of the spine in several directions. Proper spacing is important because it allows the intervertebral foramen to maintain its height, which allows the segmental nerve roots room to exit each spinal level without compression.

Further, the disc allows the spine to compress and rebound when the spine is axially loaded during such activities as jumping and running. Importantly, it also resists the downward pull of gravity on the head and trunk during prolonged sitting and standing.

Furthermore, the disc allows the spinal segment to flex, rotate, and bend to the side, all at the same time during a particular activity. This would be impossible if each spinal segment were locked into a single axis of motion.

An unhealthy disc may result in pain. One way a disc may become unhealthy is when the inner nucleus dehydrates. This results in a narrowing of the disc space and a bulging of the annular ligaments. With progressive nuclear dehydration, the annular fibers can crack and tear. Further, loss of normal soft tissue tension may allow for a partial dislocation of the joint, leading to bone spurs, foraminal narrowing, mechanical instability, and pain.

Lumbar disc disease can cause pain and other symptoms in two ways. First, if the annular fibers stretch or rupture, the nuclear material may bulge or herniate and compress neural tissues resulting in leg pain and weakness. This condition is often referred to as a pinched nerve, slipped disc, or herniated disc. This condition will typically cause sciatica, or radiating leg pain as a result of mechanical and/or chemical irritation against the nerve root.

Although the overwhelming majority of patients with a herniated disc and sciatica heal without surgery, if surgery is indicated it is generally a decompressive removal of the portion of herniated disc material, such as a discectomy or microdiscectomy.

Second, mechanical dysfunction may cause disc degeneration and pain (e.g. degenerative disc disease). For example, the disc may be damaged as the result of some trauma that overloads the capacity of the disc to withstand increased forces passing through it, and inner or outer portions of the annular fibers may tear. These torn fibers may be the focus for inflammatory response when they are subjected to increased stress, and may cause pain directly, or through the compensatory protective spasm of the deep paraspinal muscles.

This mechanical pain syndrome, unresponsive to conservative treatment, and disabling to the individuals way of life, is generally the problem to be addressed by spinal fusion or artificial disc technologies.

### SUMMARY OF THE INVENTION

Traditionally, spinal fusion surgery has been the treatment of choice for individuals who have not found pain relief for chronic back pain through conservative treatment (such as physical therapy, medication, manual manipulation, etc), and have remained disabled from their occupation, from their activities of daily living, or simply from enjoying a relatively pain-free day-to-day existence. While there have been significant advances in spinal fusion devices and surgical techniques, the procedure does not always work reliably.

Artificial discs offer several theoretical benefits over spinal fusion for chronic back pain, including pain reduction and a potential to avoid premature degeneration at adjacent levels of the spine by maintaining normal spinal motion. However, like spinal fusion surgery, surgical techniques and procedures do not always work reliably for artificial disc implantation. Thus, there remains a need for improved instrumentation and techniques for disc space preparation and artificial disc implantation.

The present invention relates generally to instruments and techniques for preparing a site between two adjacent vertebra segments to receive an artificial disc therebetween. More specifically, the present invention provides instruments for vertebral endplate preparation to receive interbody fusion devices or artificial disc implants. The instruments and techniques of the present invention have particular application, but are not limited to, direct anterior or oblique-anterior approaches to the spine.

In one embodiment the invention is an anterior method for implanting an artificial disc in an intervertebral space of a human body. The method includes inserting a midline marker in a face of a vertebral body for instrument alignment and artificial disc placement. In a specific embodiment, the placement of the disc is verified for artificial disc implantation. Verification, in one embodiment includes centering a verification instrument on the disc, inserting radiopaque pins extending from the verification instrument into the disc, visualizing, via X-ray, the radiopaque pins in the disc, and removing the verification instrument from the disc after visualization. Additional steps of the method of the invention can include inserting the midline marker in a guide of the verification instrument, and impacting a proximal end of the midline marker until the midline marker is embedded in the face of the vertebral body.

In another embodiment, the invention is a kit for implanting an artificial disc in an intervertebral space of the human body. The kit includes site preparation instruments for preparing the intervertebral space, artificial disc insertion instruments for implanting the artificial disc into the prepared intervertebral space, and a midline marker for guiding the artificial disc insertion instruments into the prepared intervertebral space. In one embodiment, the verification instrument includes a radiolucent body having a proximal end and a distal end. A handle is at the distal end of the body, and at least one radiopaque pin is at the proximal end of the body. The verification instrument can further include a guide on a surface on the body for mating with a midline marker insertion instrument. The artificial disc insertion instruments can include a distraction instrument that distracts the intervertebral space upon the passing of implants or instruments therethrough, a trial spacer insertion instrument and various trial spacer heads for assessing the size of the intervertebral space, an endplate insertion instrument for inserting endplates of the artificial disc into the intervertebral space, and a core insertion instrument for inserting a core between the endplates of the artificial disc.

In another embodiment, the invention is a verification instrument for determining a disc for artificial disc replacement. The verification instrument includes a radiolucent body, the body having a proximal end and a distal end, a handle at the distal end of the body, and least one radiopaque pin at the proximal end of the body.

In still another embodiment, the invention is a midline marker for providing instrument alignment and artificial disc placement. The midline marker includes a body element having a tapered end and an attachment end. In some embodiments thereof, at least two protrusions, parallel to each other, extend from the attachment end of the body element. In another embodiment thereof, a single protrusion extends from the attachment end of the body element.

In another embodiment, the invention is an endplate shaping device. The endplate shaping device includes a frame having a proximal end and a distal end. A handle is coupled to the proximal end of the frame. A driving mechanism is disposed within the frame. Two cutting shafts, parallel to each other, each have a proximal end and a distal end. The proximal end of each shaft is separately coupled to a pivot block on the driving mechanism and is rotatable around its point of attachment. The distal end of each cutting shaft extends from the distal end of the frame. Each of a pair of cutter blades are coupled to a respective distal end of each cutting shaft.

In still another embodiment, the invention is a distraction instrument that includes a body element, a pair of diametrically opposing arms coupled to the body, at least one arm including a midline marker guide, a distraction mechanism coupled between the diametrically opposing arms, and a handle coupled to the distraction mechanism.

In yet another embodiment, the invention is an endplate insertion instrument. The endplate insertion instrument includes a body element, a pair of diametrically opposing arms coupled to the body, the arms having first and second opposed surfaces respectively having first and second opposed alignment surfaces (such as first and second opposed grooves), an endplate holder coupled to one end of each arm, a handle portion coupled to an opposite end of each arm and a mounting plate, each arm slidably coupled to opposite ends of the mounting plate.

In another embodiment, the invention is a core insertion instrument. The core insertion instrument includes a body having a handle end and an insertion end. The core insertion also includes a pair of diametrically opposing guides on opposing surfaces of the insertion end.

In still another embodiment, the invention includes trial spacer head for determining a correct-sized artificial disc. The trial spacer head includes a body element having superior and inferior surfaces. Also included are diametrically opposing grooves on the superior and inferior surfaces of the body, and radiopaque pins within the radiolucent body for x-ray visualization.

The invention has many advantages. For example, the invention provides reliably correct alignment for preparing a disc space of artificial disc implantation. The invention also provides the reliably correct alignment for artificial disc insertion into the prepared disc space.

### BRIEF DESCRIPTION OF THE DRAWINGS

FIG. 1A shows a perspective view of the lower spine, highlighting a surgically prepared disc space;

FIG. 1B shows a perspective view of one embodiment of a disc verification instrument of the invention which can be used to verify the surgical level and mark the midline of the surgical level;

FIG. 2A shows a perspective view of one embodiment of a distraction instrument of the invention inserted into the intervertebral space of the lower spine;

FIG. 2B shows a perspective view of one embodiment of a trial spacer of the invention being inserted into the intervertebral space using the distraction instrument as a guide;

FIG. 2C shows an anterior view of the distraction instrument and the trial spacer of FIG. 2B inserted into the intervertebral space;

FIG. 2D shows a perspective view of the trial spacer inserted into the intervertebral space;

FIG. 2E shows another perspective view of the trial spacer inserted into the intervertebral space.

FIG. 2F is a perspective view of trial spacer inserted into the intervertebral space.

FIG. 3A shows a perspective view of one embodiment of the midline marker of the invention being inserted into a face of a vertebra;

FIG. 3B shows a perspective view of the midline marker inserted into the face of the vertebra;

FIG. 4A shows a perspective view of a cutting end of one embodiment of an endplate shaping instrument of the invention;

FIG. 4B shows a perspective view of the endplate shaping instrument inserted into the intervertebral space using the midline marker as a guide;

FIG. 5A shows a perspective view of an endplate insertion end of one embodiment of an endplate insertion instrument of the invention, highlighting superior and inferior endplates;

FIG. 5B shows a perspective view of the endplate insertion instrument of FIG. 5A inserted into the intervertebral space in a closed position using the distraction instrument as a guide;

FIG. 5C shows a perspective view of the endplate insertion instrument of FIG. 5B inserted into the intervertebral space in an open position;

FIG. 6A shows a perspective view of a polyethylene core loaded on one embodiment of a core insertion instrument of the invention;

FIG. 6B shows a perspective view of the core insertion instrument of FIG. 6A being inserted into the intervertebral space using the endplate instrument as a guide;

FIG. 6C shows a perspective view of the endplates and core of FIG. 6B inserted into the intervertebral space;

FIG. 7A shows a perspective view of a core retention clip loaded onto a retention clip insertion instrument of the invention;

FIG. 7B shows a perspective view of the completed artificial disc inserted into the intervertebral space;

FIG. 8A shows a perspective view of one embodiment of a distraction instrument of the invention;

FIG. 8B shows a side view of the distraction instrument of FIG. 8A;

FIG. 8C shows a superior view of the distraction instrument of FIG. 8A;

FIG. 8D shows a perspective view of another embodiment of a distraction instrument of the invention;

FIG. 8E shows a perspective view of another embodiment of a distraction instrument of the invention;

FIG. 9A shows a superior view of one embodiment of a trial spacer insertion instrument of the invention;

FIG. 9B shows a side view of the trial spacer insertion instrument of FIG. 9A;

FIG. 9C shows a perspective view of another embodiment of a trial spacer insertion instrument of the invention

FIG. 10A shows a perspective view of one embodiment of a trial spacer head;

FIG. 10B shows a superior view of the trial spacer head of FIG. 10A;

FIG. 10C shows a rear view of the trial spacer head of FIG. 10A;

FIG. 10D shows a side view of the trial spacer head of FIG. 10A;

FIG. 10E shows a perspective view of another embodiment of a trial spacer head of the invention;

FIG. 10F shows a superior view of the trial spacer head of FIG. 10E;

FIG. 11A shows a perspective view of one embodiment of a midline marker insertion instrument of the invention;

FIG. 11B shows a perspective view of another embodiment of a midline marker insertion instrument of the invention;

FIG. 12A shows a perspective view of one embodiment of a midline marker of the invention;

FIG. 12B shows a superior view of the midline marker of FIG. 12A;

FIG. 12C shows a side view of the midline marker of FIG. 12A;

FIG. 12D shows a perspective view of another embodiment of a midline marker of the invention;

FIG. 13A shows a perspective view of one embodiment of an endplate shaping instrument of the invention;

FIG. 13B shows an inferior view of the endplate shaping instrument of FIG. 13A;

FIG. 13C shows a side view of the endplate shaping instrument of FIG. 13A;

FIG. 13D shows a perspective view of one embodiment of a shaft spreader of the endplate shaping instrument of FIG. 13A;

FIG. 13E shows a side view of the shaft spreader of FIG. 13D;

FIG. 14A shows a perspective view of one embodiment of an endplate insertion instrument of the invention;

FIG. 14B shows an exploded view of the endplate insertion instrument of FIG. 14A;

FIG. 14C shows an inferior view of the endplate insertion instrument of FIG. 14A;

FIG. 14D shows a side view of the endplate insertion instrument of FIG. 14A;

FIG. 14E shows a perspective view of another embodiment of the endplate insertion instrument of the invention;

FIG. 15A shows a perspective view of one embodiment of a core insertion instrument of the invention;

FIG. 15B shows a superior perspective view of a cassette of the core insertion instrument of FIG. 15A;

FIG. 15C shows an inferior perspective view of the cassette of the core insertion instrument of FIG. 15A;

FIG. 15D shows a superior view of one embodiment of an insertion shaft of the core insertion instrument of FIG. 15A;

FIG. 15E shows a perspective view of another embodiment of the core insertion instrument of the invention;

FIG. 16 shows a perspective view of one embodiment of a retention clip insertion instrument of the invention;

FIG. 17 shows a perspective view of one embodiment of a retention clip removal instrument of the invention;

FIG. 18 shows a perspective view of another embodiment of a verification instrument of the invention;

FIG. 19 is a perspective view of an endplate inserter and spreader providing distraction and core trialing;

FIG. 20 is a perspective view of a first core height trial instrument;

FIG. 21 is a perspective view of a second core height trial instrument;

FIG. 22 is a perspective view of an endplate insertion instrument in a closed position;

FIG. 23 is a perspective view of an endplate insertion instrument in an open position; and

FIG. 24 is a perspective view of a spreader.

### DETAILED DESCRIPTION OF THE INVENTION

The foregoing and other objects, features and advantages of the invention will be apparent from the following more particular description of preferred embodiments of the invention, as illustrated in the accompanying drawings in which like reference characters refer to the same parts throughout the different views. The same number appearing in different drawings represents the same item. The drawings are not necessarily to scale, with emphasis instead being placed upon illustrating the principles of the invention.

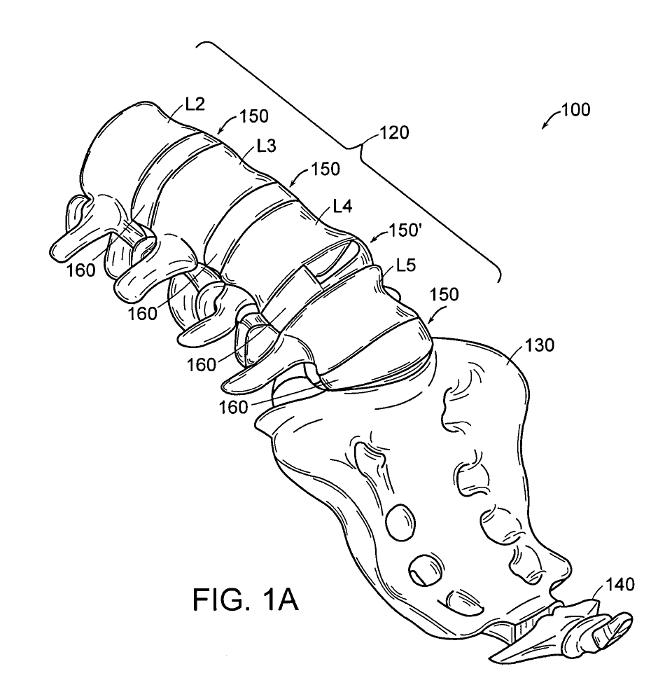

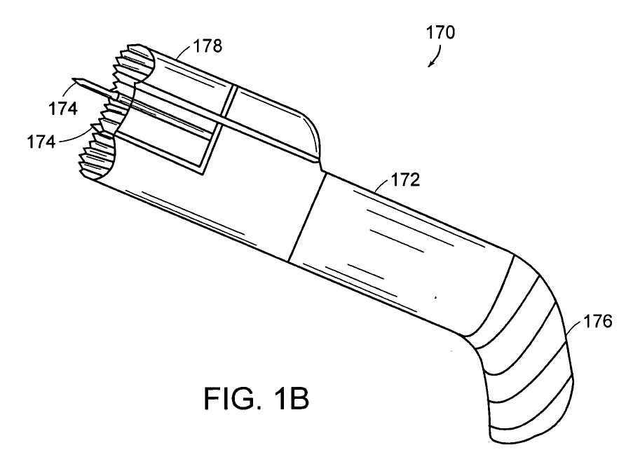

In general, the surgical procedure for implantation utilizes an anterior approach. During the surgery, a small incision is made in the abdomen below the belly button. The organs are carefully moved to the side so the surgeon can visualize the spine. The surgeon then removes a portion of a disc. In one embodiment, the implant is inserted; endplates first followed by the polyethylene core. The disc stays in place from the tension in spinal ligaments and the remaining part of the annulus of the disc. In addition, compressive forces of the spine keep the disc in place. A successful implantation is governed by good patient selection, correct artificial disc size selection, and proper artificial disc positioning. To that end, a method for proper artificial disc positioning is described with respect to FIGS. 1-7B.

In another embodiment, the entire implant assembly (e.g., both prosthetic endplates and its core) is inserted simultaneously.

FIG. 1A shows a perspective view of the lower region of spine 100. This region comprises lumbar spine 120, sacral spine 130, and coccyx 140. Lumbar spine 120 is comprised of five (5) vertebrae L5, L4, L3, L2, and L1 (not shown). Intervertebral discs 150 link contiguous vertebra from C2 (not shown) to sacral spine 130, wherein a single quotation (‘) denotes a damaged disc, for example 150′.

Intervertebral disc 150 is comprised of a gelatinous central portion called the nucleus pulposus (not shown) and surrounded by an outer ligamentous ring called the annulus fibrosus (“annulus”) 160. The nucleus pulposus is composed of 80-90% water. The solid portion of the nucleus is Type II collagen and non-aggregated proteoglycans. Annulus 160 hydraulically seals the nucleus, and allows intradiscal pressures to rise as the disc is loaded. Annulus 160 has overlapping radial bands which allow torsional stresses to be distributed through the annulus under normal loading without rupture.

Annulus 160 interacts with the nucleus. As the nucleus is pressurized, the annular fibers prevent the nucleus from bulging or herniating. The gelatinous nuclear material directs the forces of axial loading outward, and the annular fibers help distribute that force without injury.

Damaged disc 150′ is prepared to receive the artificial disc by removing a window the width of the artificial disc to be implanted from annulus 160 of damaged disc 150′. The nucleus pulposus of disc 150′ is completely removed.

Damaged disc 150′ can be verified using a disc verification instrument 170 shown in FIG. 1B. Verification instrument 170 includes radiolucent body 172, radiopaque pins 174, handle 176, and guide 178. Before preparing damaged disc 150′, a surgeon may want to determine he has correctly chosen damaged disc 150′. To do so, the surgeon inserts radiopaque pins 174 into damaged disc 150′ (FIG. 1A) using handle 176 of verification instrument 170. Damaged disc 150′ can be visualized via X-ray utilizing radiopaque pins 174 within and extending from verification instrument 170. Verification instrument 170 also provides a centerline for preparing damaged disc 150′ by providing a visual marker that can be compared to the local bony anatomy. Verification instrument 170 further provides midline marker guide 178 for optionally impacting a midline marker into a surface of the vertebral body. The midline marker will be discussed in more detail below.

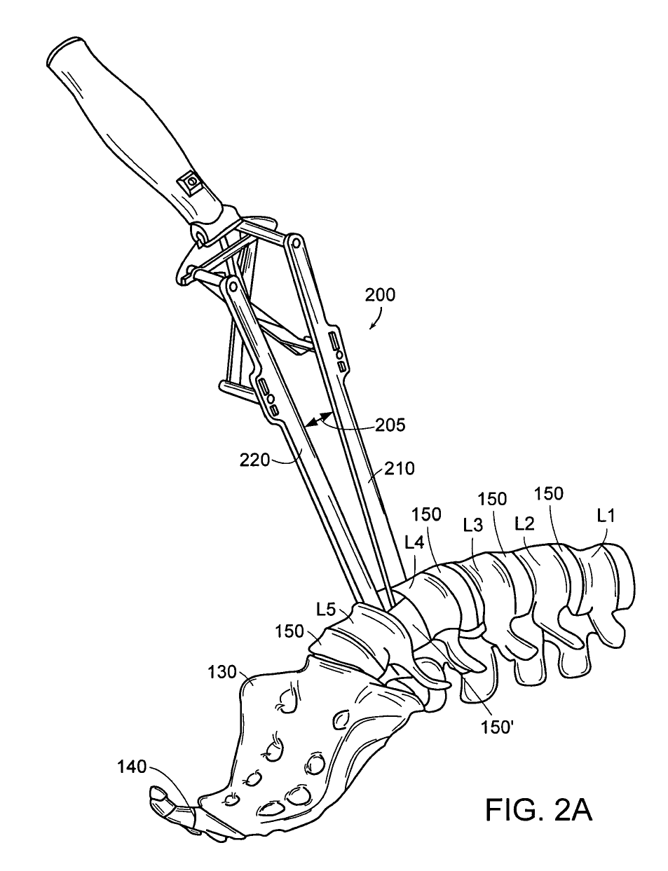

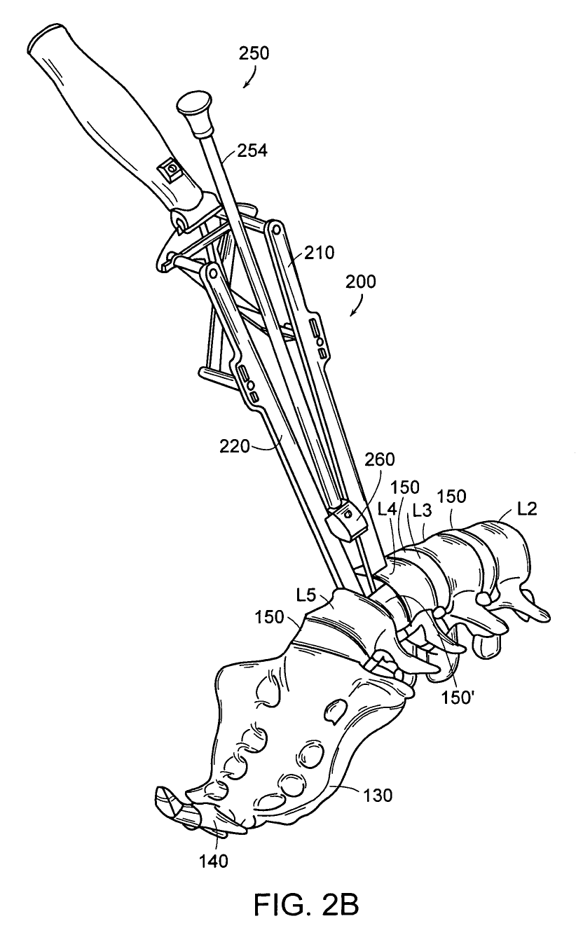

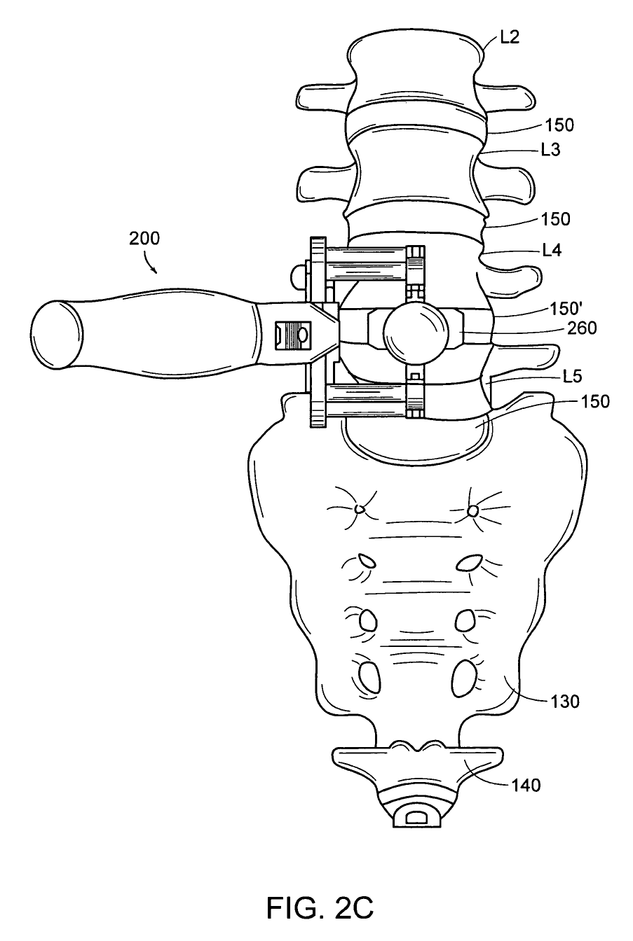

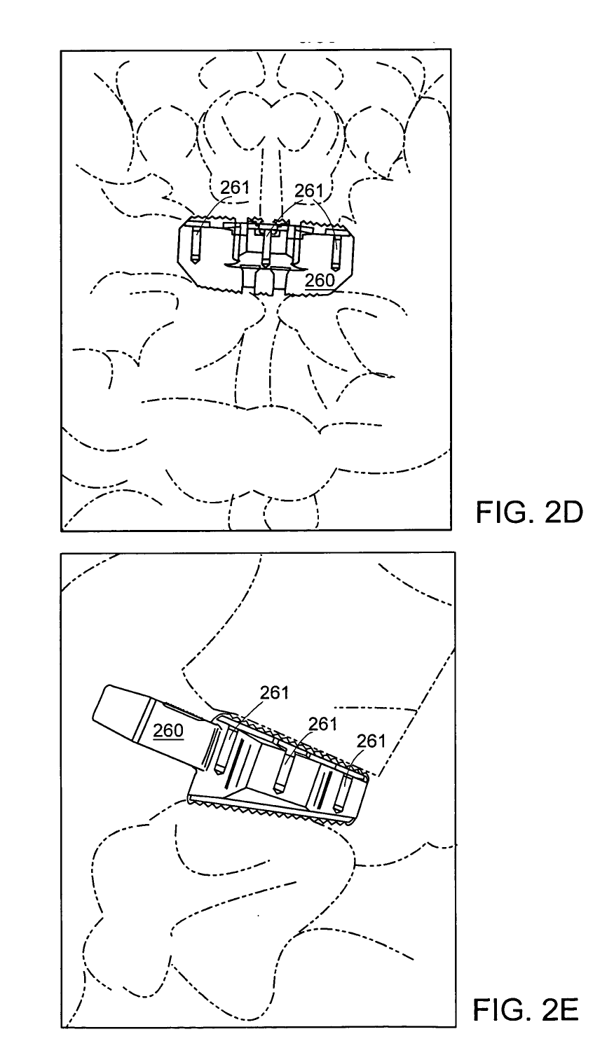

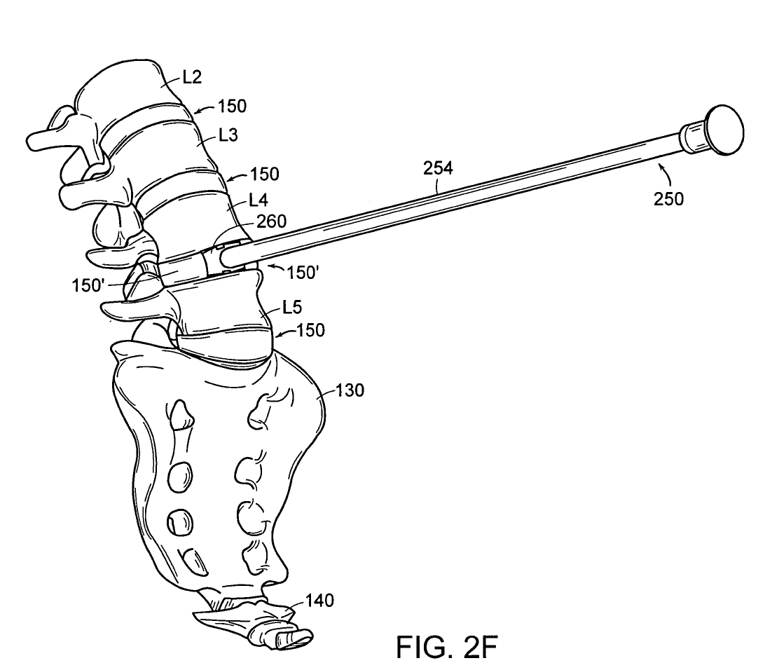

As shown in FIG. 2A, distraction instrument 200 is shown fully inserted into the prepared intervertebral space. Distraction instrument 200 operates in two positions, a closed position (not shown) for insertion into the intervertebral space and open position 205 for distraction of the intervertebral space. As shown in open position 205 of FIGS. 2A-2C, distraction instrument 200 distracts the intervertebral space to a given distance upon insertion of any one of trial spacers 260 (FIGS. 2B and 2C), artificial disc implants, or spinal fusion cages.

Trial spacers 260 are used to determine an appropriate size of the artificial disc implant. The surgeon selects an appropriate sized trial spacer 260 from a kit of trial spacers. The kit of trial spacers 260 can include about 60 discrete sizes ranging from 10 mm, ON, extra small to 14 mm, 15N, extra large. Trial spacers 260 are made of colored acetal copolymers, such as Celcon®, and have three metallic markers which relate the true position of the trial during intra-operative imaging. In some embodiments, about 28 to about 40 discrete sizes are provided in the kit, are made of a composite comprising a radiolucent material (such as RadelR) and have four metallic markers.

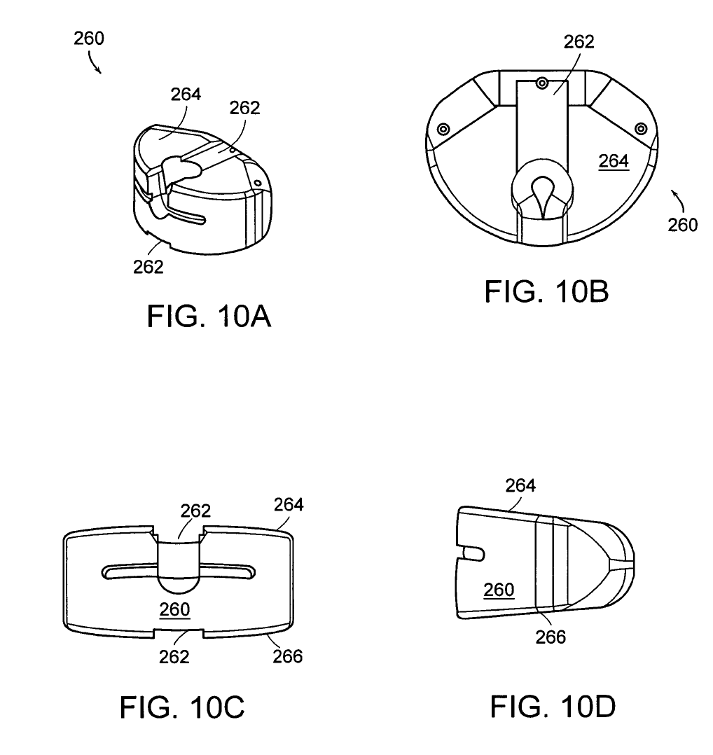

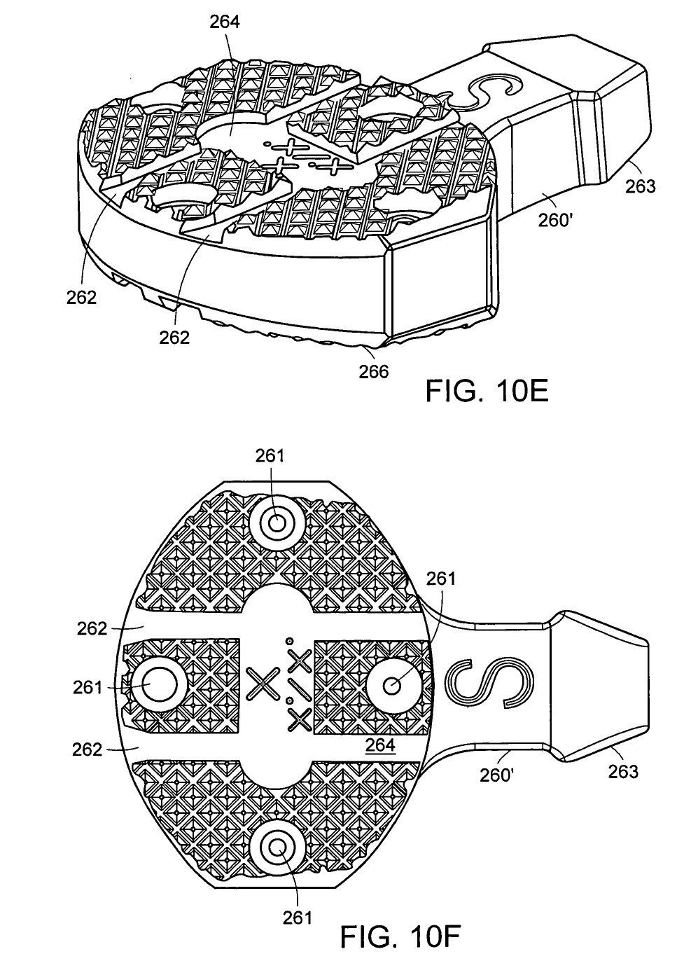

With reference to FIGS. 2B-2E and 10A-10D, the selected trial spacer 260 is passed down superior 210 and inferior 220 arms of distraction instrument 200 using trial spacer insertion instrument 250. Groove 262 on the superior and inferior faces 264, 266 (FIGS. 10A-10d) of trial spacer 260 allow trial spacer 260 to maintain a centered position on arms 210, 220 of distraction instrument 200 while being guided into the intervertebral space. The intervertebral space becomes increasingly distracted the closer trial spacer 260 gets to the intervertebral space to allow for easier insertion of trial spacer 260 into the intervertebral space. The trial placement can be visualized via X-ray utilizing radiopaque markers 261 (FIGS. 2D and 2E) within a radiolucent head of trial spacer 260. Three of fours pins 261 are visible on the x-ray if trial spacer 260 is positioned correctly. Radiolucent head 260 may also be treated with a radiopaque agent to visualize head 260 within the intervertebral space. The surgeon repeats this step, as necessary, until the appropriate size of the artificial disc implant is determined.

As shown in FIG. 2F, once the appropriate sized trial spacer 260 has been determined, distraction instrument 200 is removed and the remaining instruments can be properly setup based on the appropriate sized artificial disc implant.

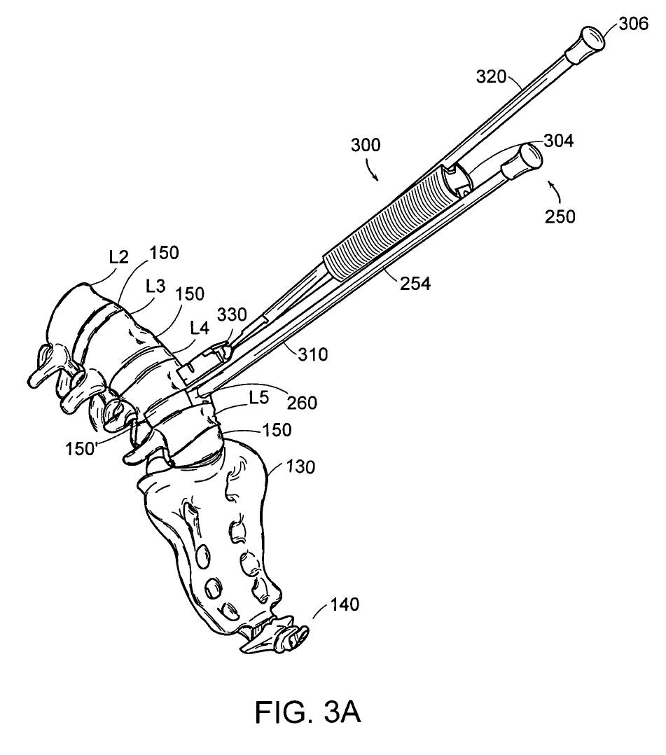

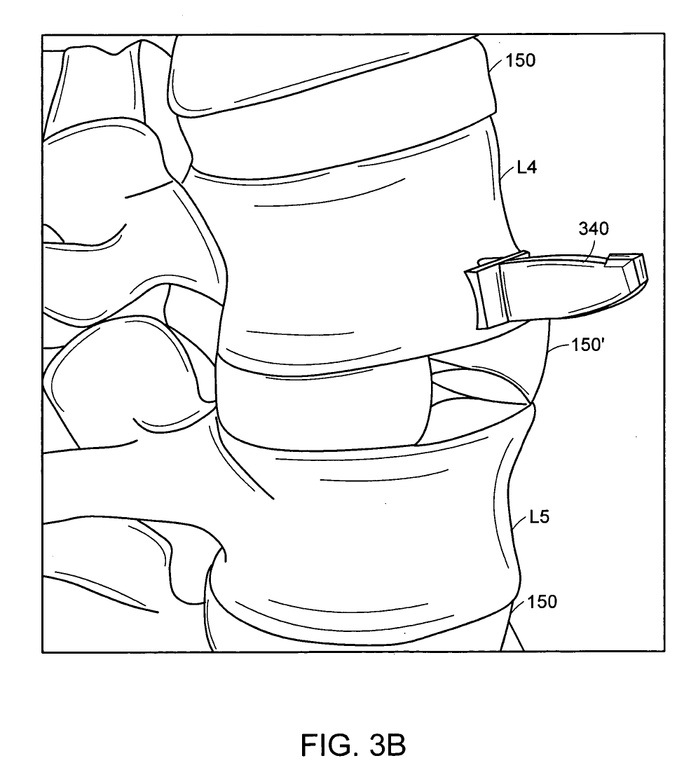

As shown in FIGS. 3A and 3B, midline marker insertion instrument 300 captures the shaft of trial spacer insertion instrument 250. Additionally, a horizontal notch on the tip 330 of midline marker insertion instrument 300 mates with a horizontal slot in trial spacer 260 to provide proper orientation. Once alignment and orientation have been verified, midline marker 340 is impacted into a face of the vertebral body. In one embodiment, midline marker 340 is positioned slightly superior to the superior vertebral endplate of the intervertebral space.

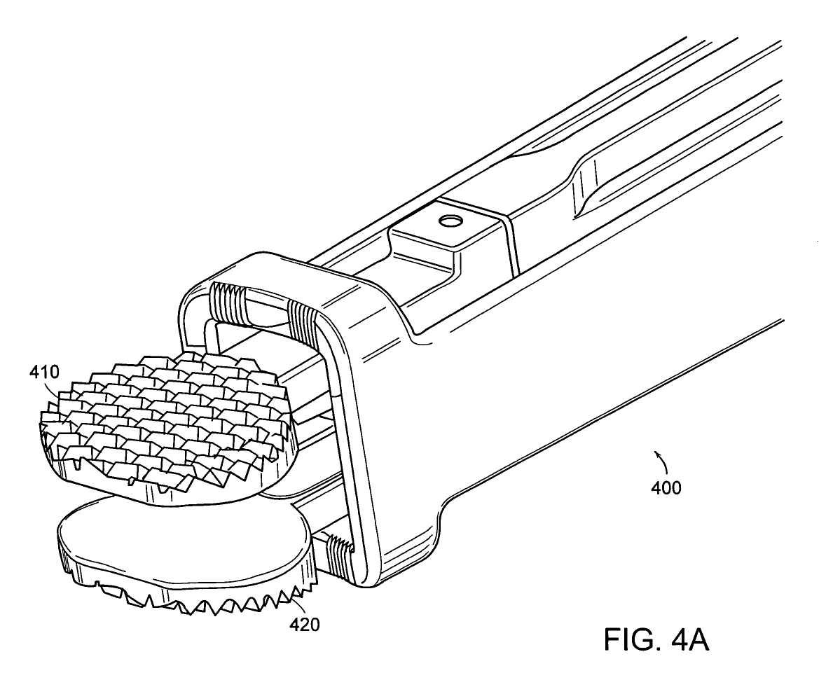

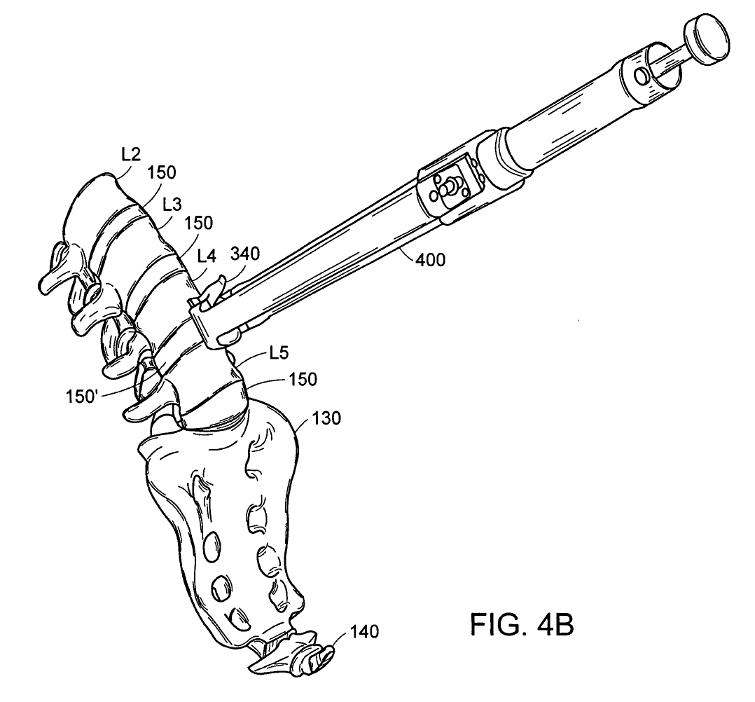

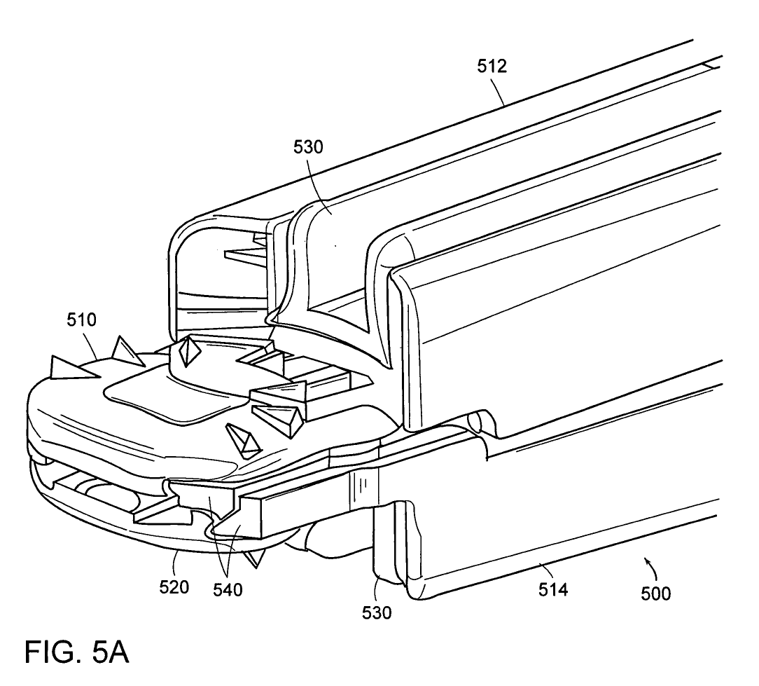

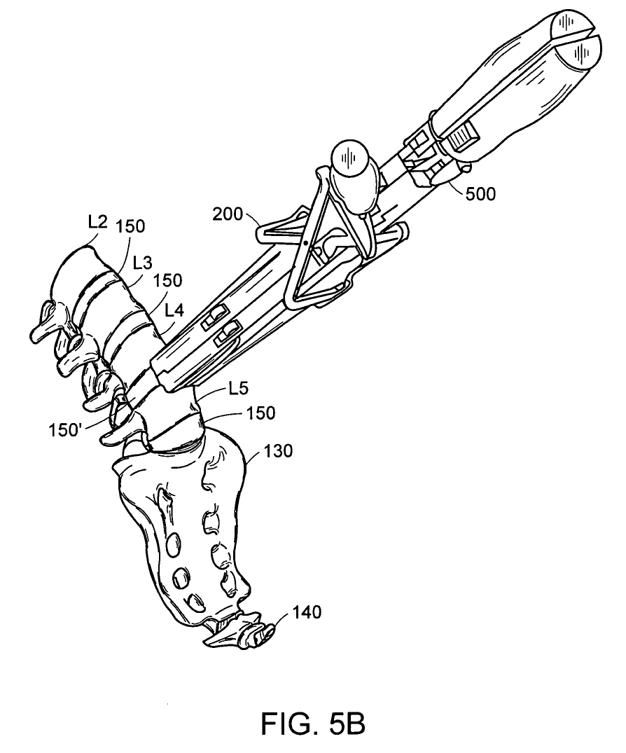

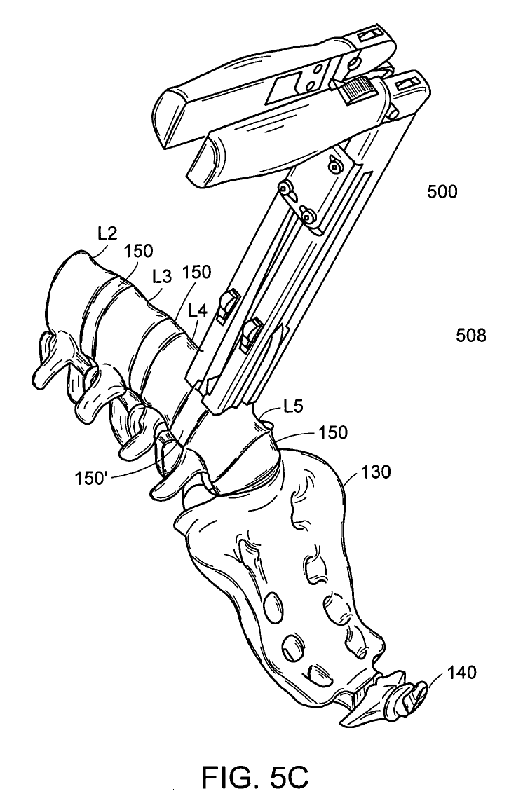

As shown in FIGS. 4A and 4B, optional endplate shaping tool 400 can be used to shape vertebral bodies to conform to the shape of the artificial disc if desired. Endplate shaping tool 400 is inserted into the intervertebral space. The placement of endplate shaping tool 400 is keyed off midline marker 340. Endplate shaping tool employs superior cutting surface 410 and inferior cutting surface 420. Cutting surfaces 410, 420 shape endplates 510, 520 (FIG. 5A) and augment the contact area between the artificial disc and the anatomy. Cutting surfaces 410, 420 are contoured to match the contour of the external faces of endplates 510, 520 of the artificial disc. Cutting is performed with a mechanically driven, oscillatory motion having a short stroke. It is understood by one skilled in the art that a hand operated endplate shaping tool employing cutting blades as described above may be used.

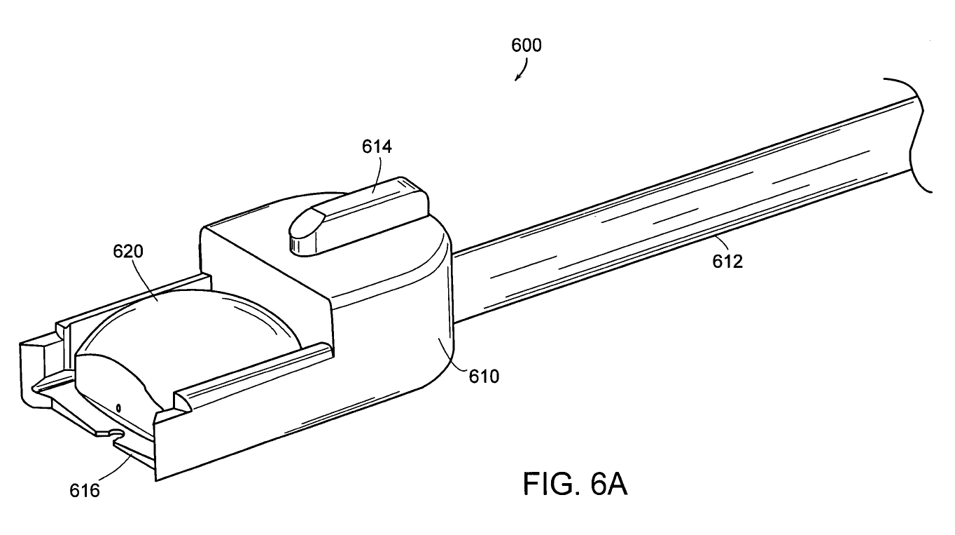

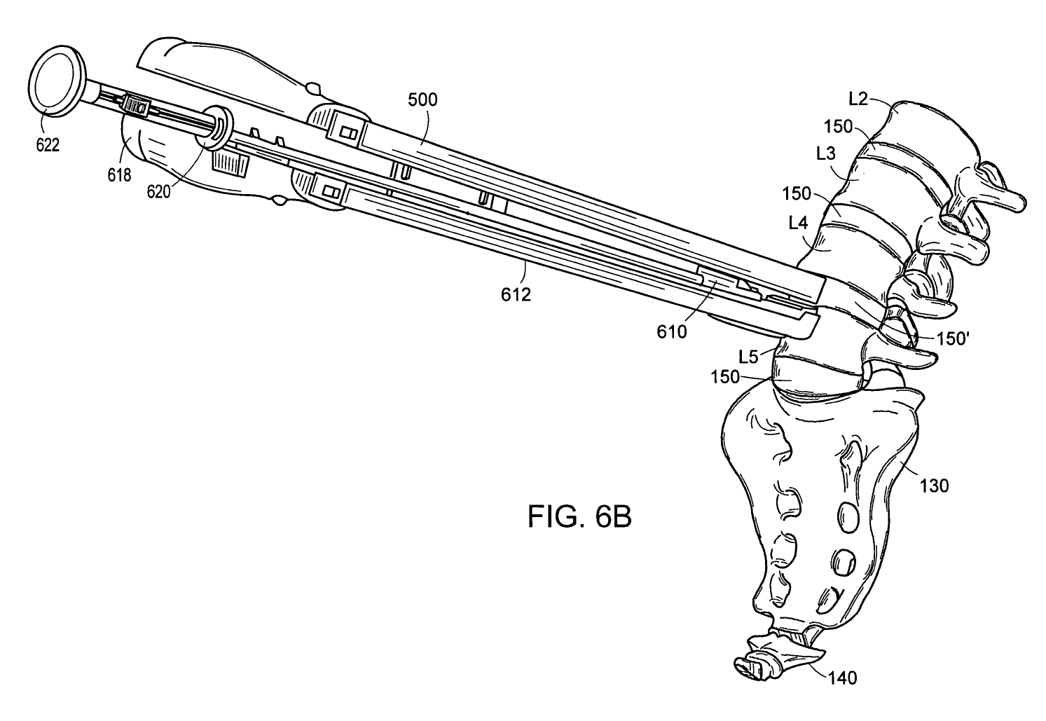

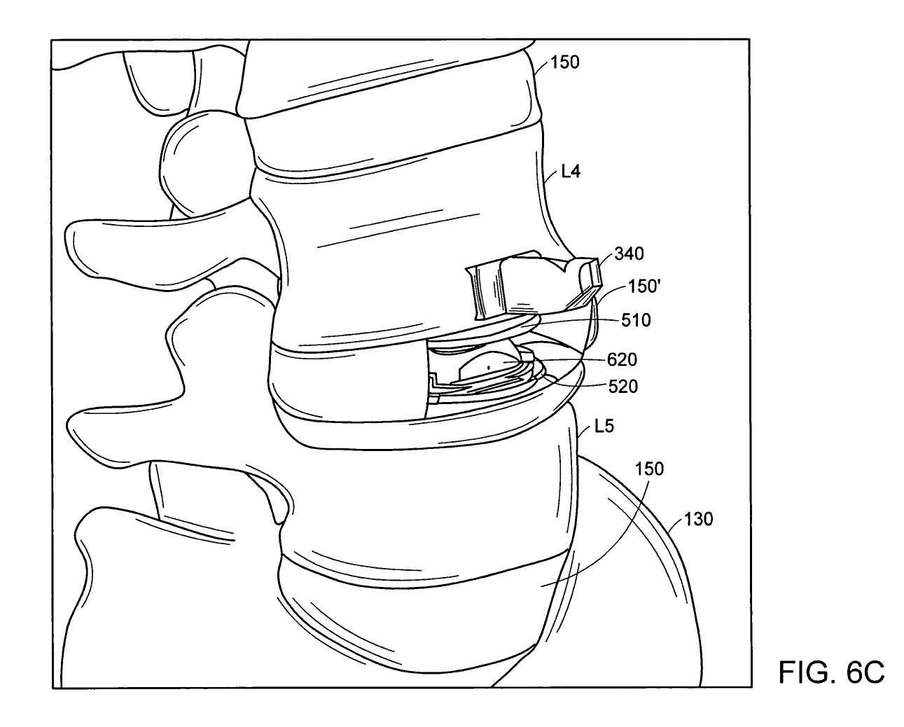

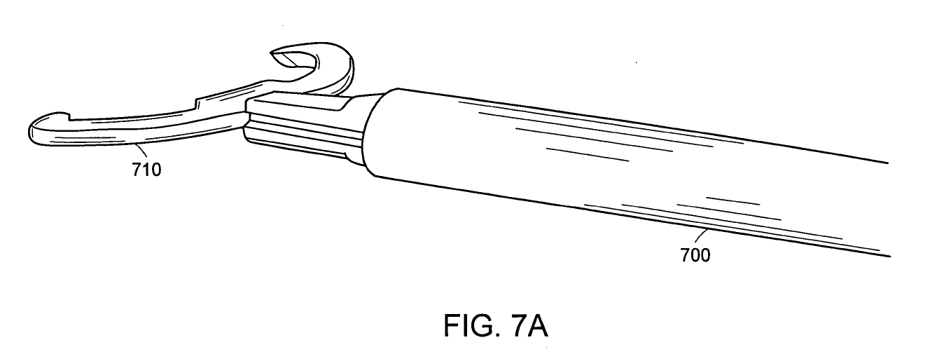

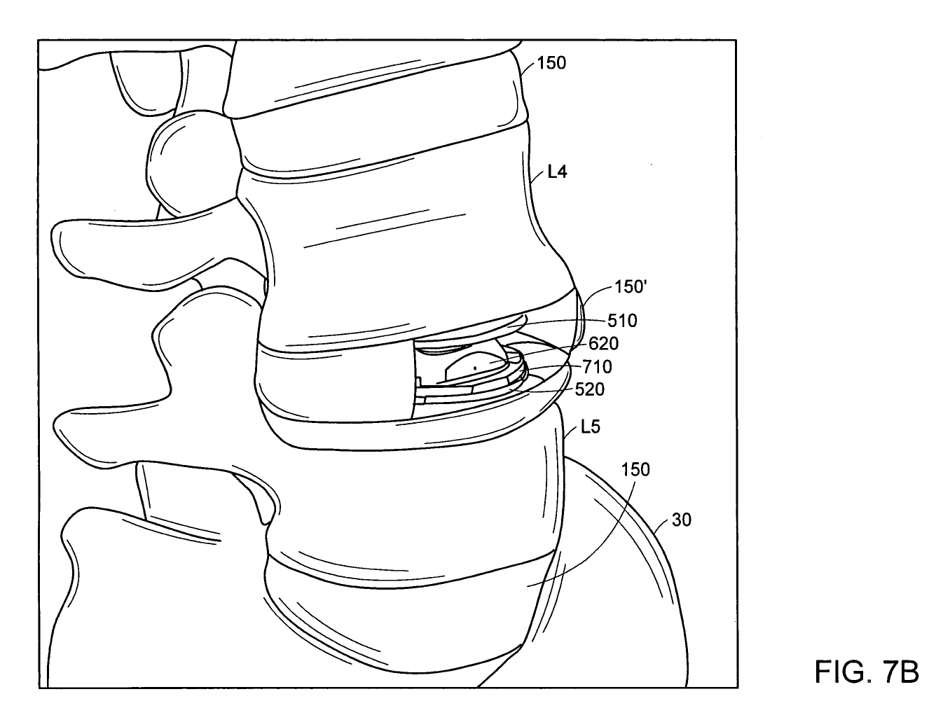

With reference to FIGS. 5A-5C, an artificial disc includes superior endplate 510, inferior endplate 520, polyethylene core 620 (FIG. 6A), and retention clip 710 (FIGS. 7A and 7B).

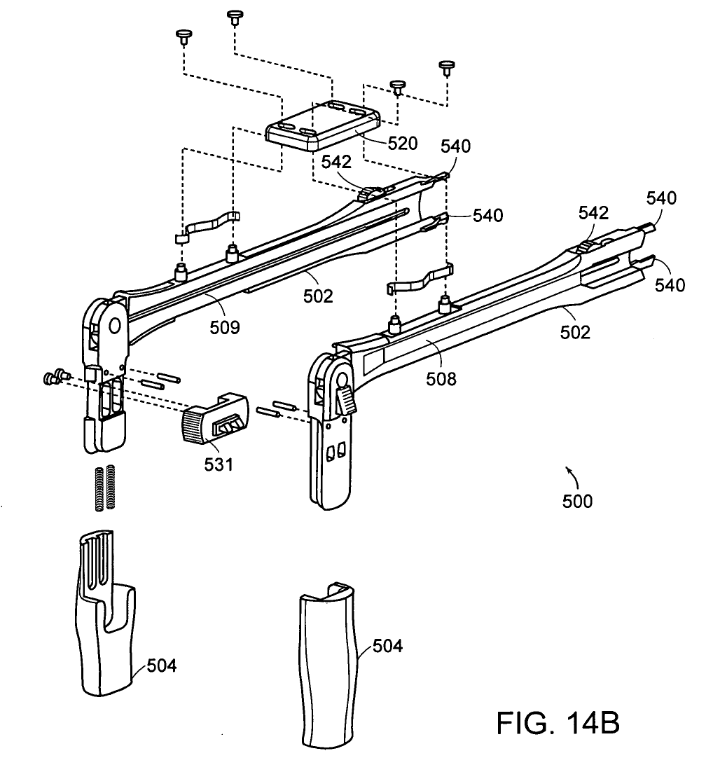

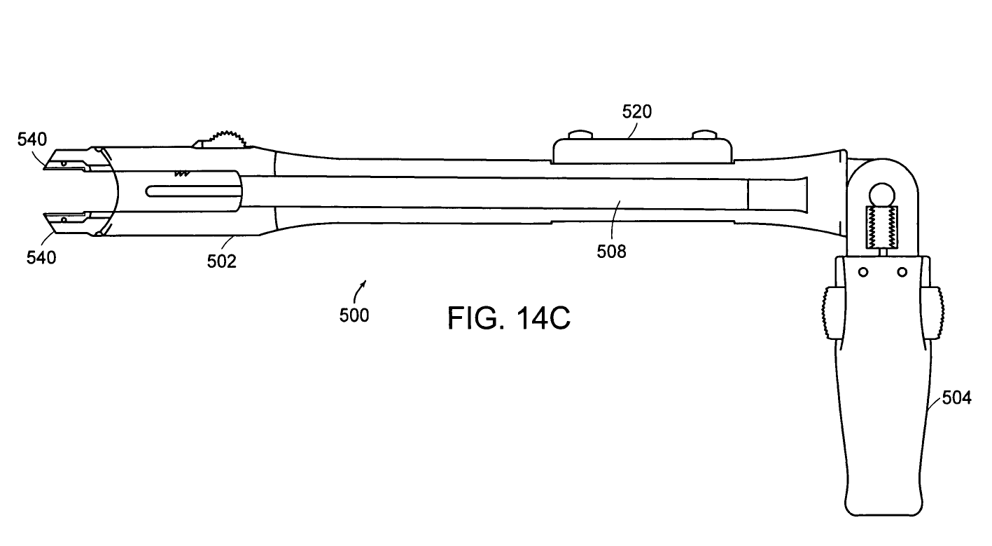

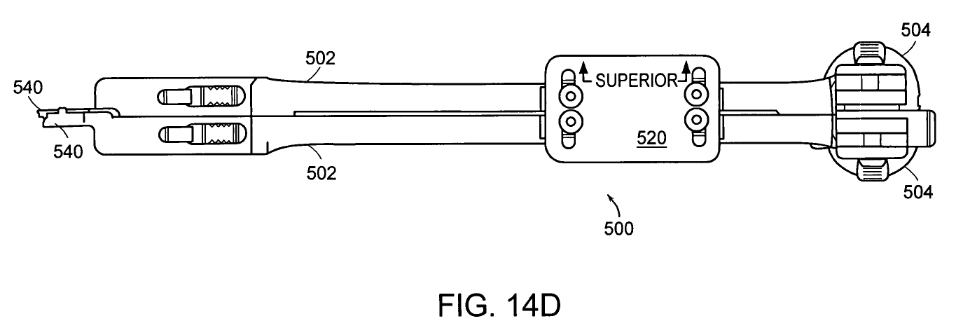

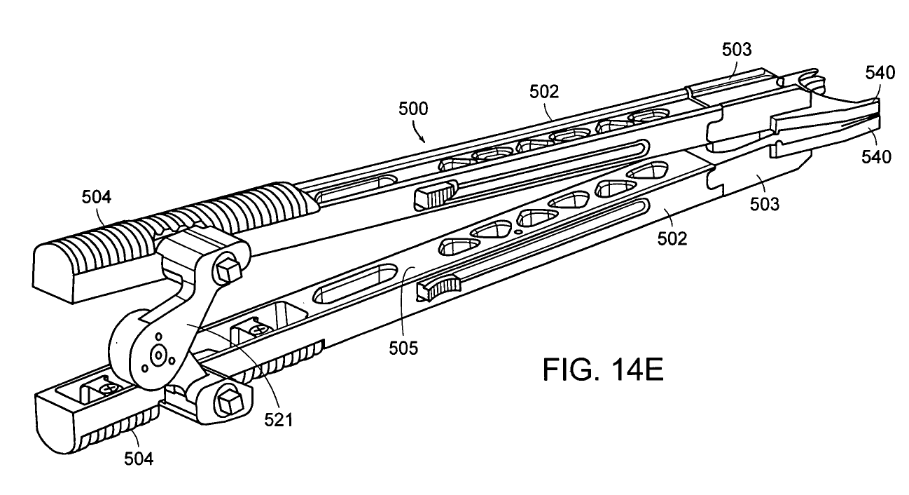

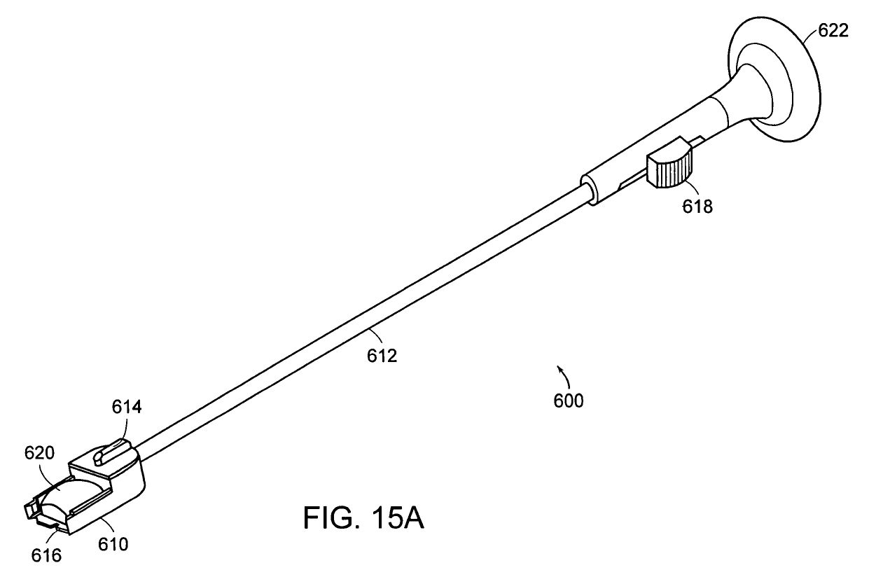

As shown in FIGS. 5A, 5B, and 5C, superior and inferior endplates 510, 520 are loaded onto tines 540 of endplate insertion instrument 500. Endplate insertion instrument 500 holds endplates 510, 520 in proper orientation in close proximity to each other, without the polyethylene core. Distraction instrument 200 (FIG. 5B) is reinserted into the intervertebral space. Midline marker 340 (FIG. 3B) recesses into the other face of superior arm 210 (FIG. 2A) of distraction instrument 200, retaining instrument alignment. Endplate insertion instrument 500 (FIG. 5A) is passed down distraction instrument 200. Slot 508 (FIGS. 14A-14C) on the superior and interior faces 512, 514 of endplate insertion instrument 500 mate with superior and inferior arms 210, 220 (FIGS. 2A and 2B) of distraction instrument 200 to maintain alignment. Endplates 510, 520 are driven towards the surgical site thereby initiating primary distraction. Artificial disc insertion depth is controlled by interchangeable spacers 530 in endplate insertion instrument 500 which comes to rest upon the external boney vertebral face when proper depth is obtained. Distraction instrument 200 is removed from the intervertebral space once endplate insertion is completed. Endplate insertion instrument 500 is opened allowing endplates 510, 520 to engage the vertebral endplates.

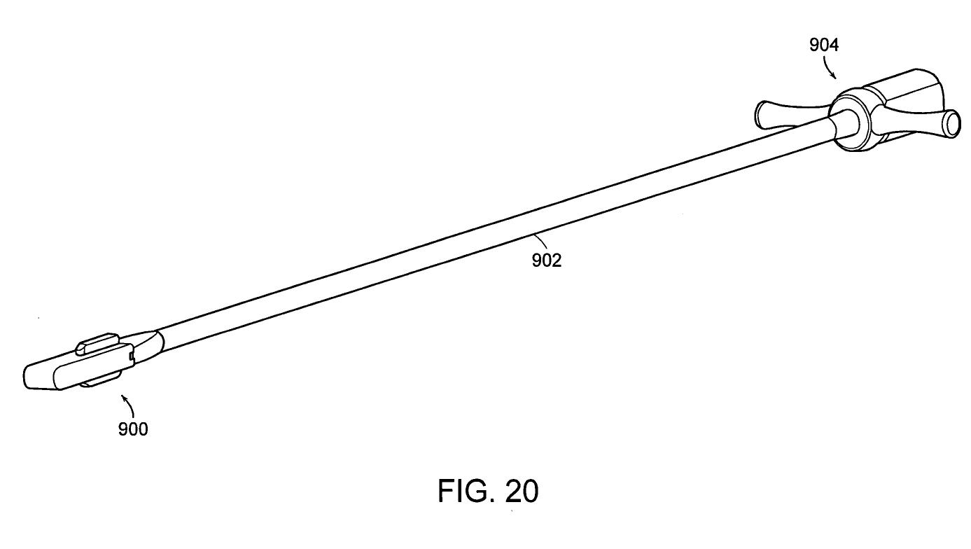

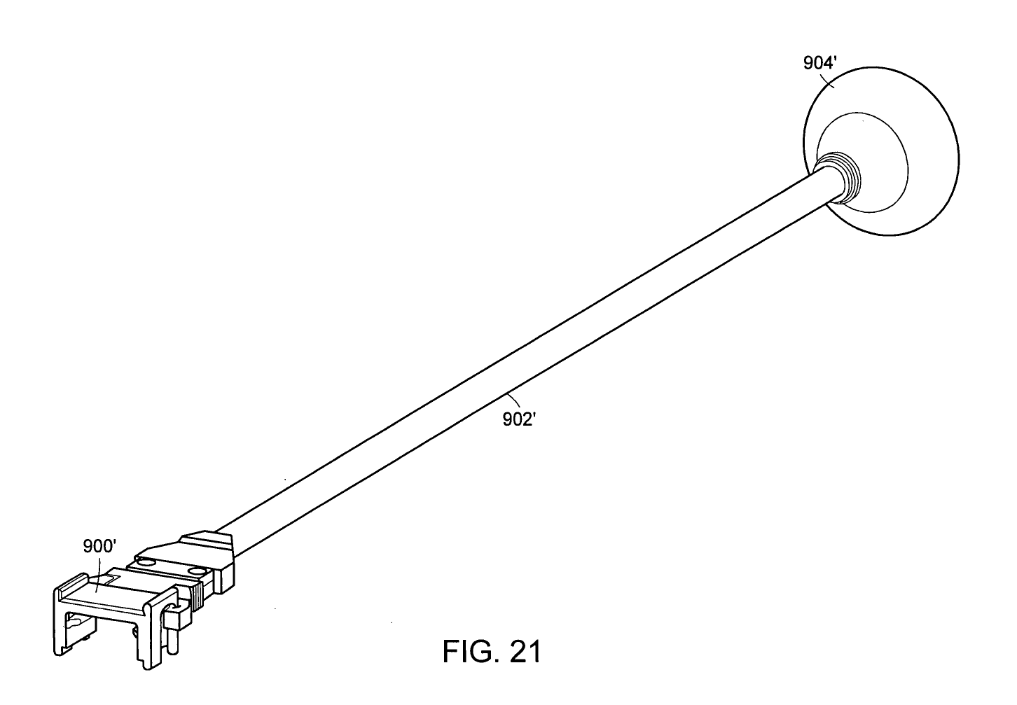

As shown in FIGS. 6A and 6B, following the insertion of endplates 510, 520 (FIG. 5A), core 620 is inserted between endplates 510, 520 with core insertion instrument 600. After the prosthetic endplates are put in place, the appropriate height of the core implant can be determined by attaching core height trial 613 to an inserter rod and inserting the trial into the disc space (FIGS. 20 and 21). Core insertion instrument 600 provides the following functions: (1) house, protect, and deliver core 620; (2) provide final distraction; and (3) indicate to the surgeon the height of core 620 being inserted. Core insertion instrument 600 includes the following components: 1) disposable cassette 610 and 2) cannulated shaft 612. Cannulated shaft 612 includes a pushrod (not shown) used to push core 620 into its final placement. Cassette 610 has fins 614 on its superior and inferior surfaces. Fins 614 key into slots 509 (FIG. 14B) located in the center of endplate insertion instrument 500. This alignment keeps core 620 centered with respect to endplates 510, 520 (FIG. 5A). As cassette 610 rides down endplate insertion instrument 500, endplates 510, 520 are distracted to a height that will allow for polyethylene core 620 to be inserted. Cassette 610 comes to its stopping point when its face 616 rests upon rails (not shown) located on the superior face (not shown) of inferior endplate 520. Thumb piece 618 at handle end 622 of core insertion instrument 600 is used to gently move core 620 from cassette 610 into its final position in the intradiscal space. Endplate insertion instrument 500 and core insertion instrument 600 are removed from the surgical site to leave only midline marker 340, and artificial disc components (510, 520, 620) as shown in FIG. 6C.

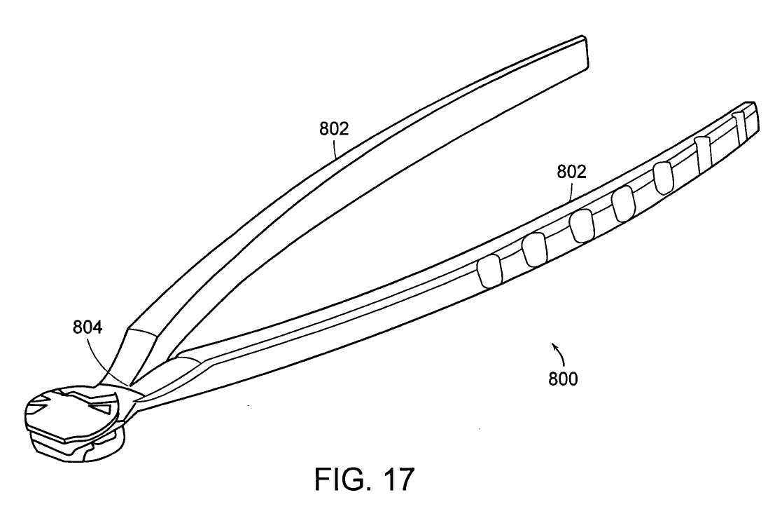

As shown in FIGS. 7A and 7B, retention clip 710 is placed on superior face of inferior endplate 520 to anteriorly secure polyethylene core 620 between endplates 510, 520. Retention clip 710 can be made from titanium or any material known in the art for securing core 620 between endplates 510, 520. Retention clip 710 is placed attached using retention clip insertion instrument 700. Retention clip 710 slides down the rails of the artificial disc and snaps into place. Midline marker 340 is removed and the procedure is completed. Retention clip 710 can be removed after installation using retention clip remover 800 (FIG. 17). Retention clip 710 may need to be removed to replace polyethylene core 620 due to damage or the surgeon's preference. Retention clip remover 800 is designed to fit within the tight constraints of the intradiscal space. Retention clip remover 800 uses small arms designed to fit between retention clip 710 and core 610 to splay the arms of retention clip 710 and allow for removal.

The above-described method can be accomplished with the instruments described in further detail below.

Distraction Instrument

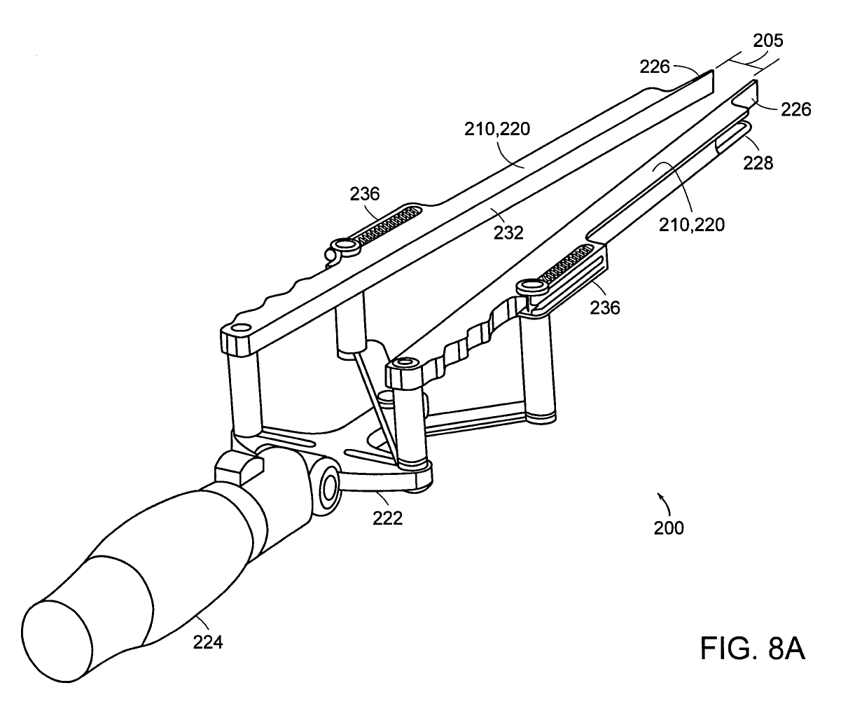

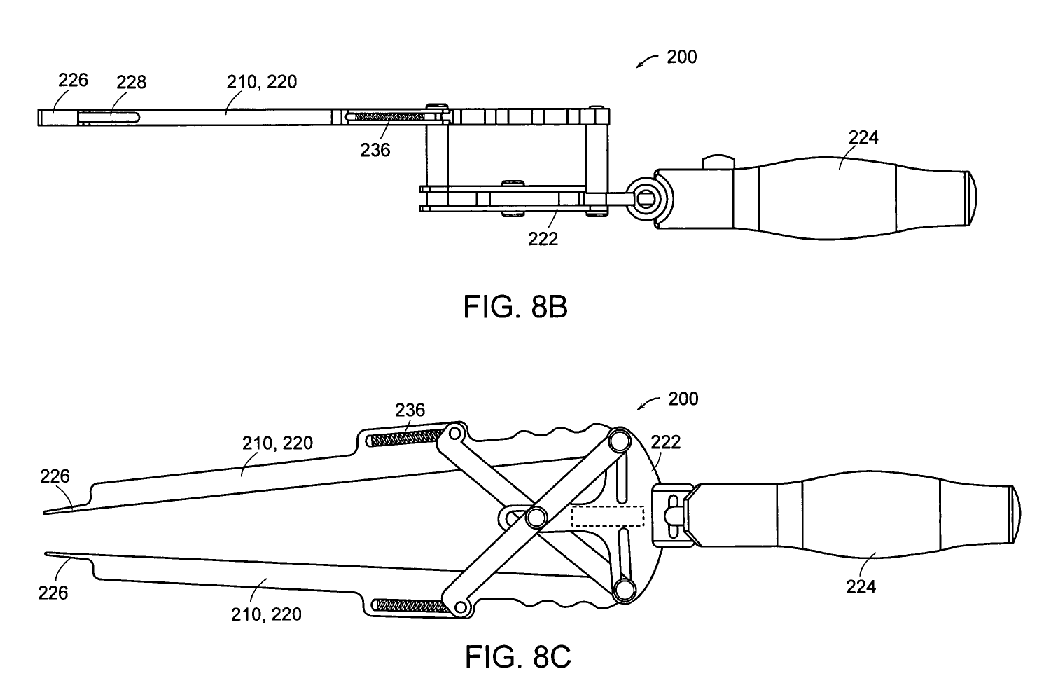

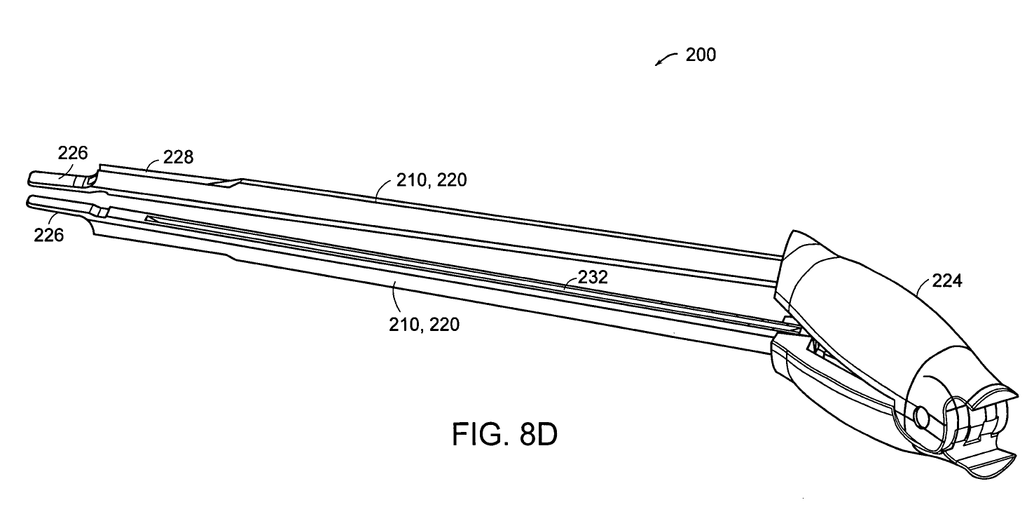

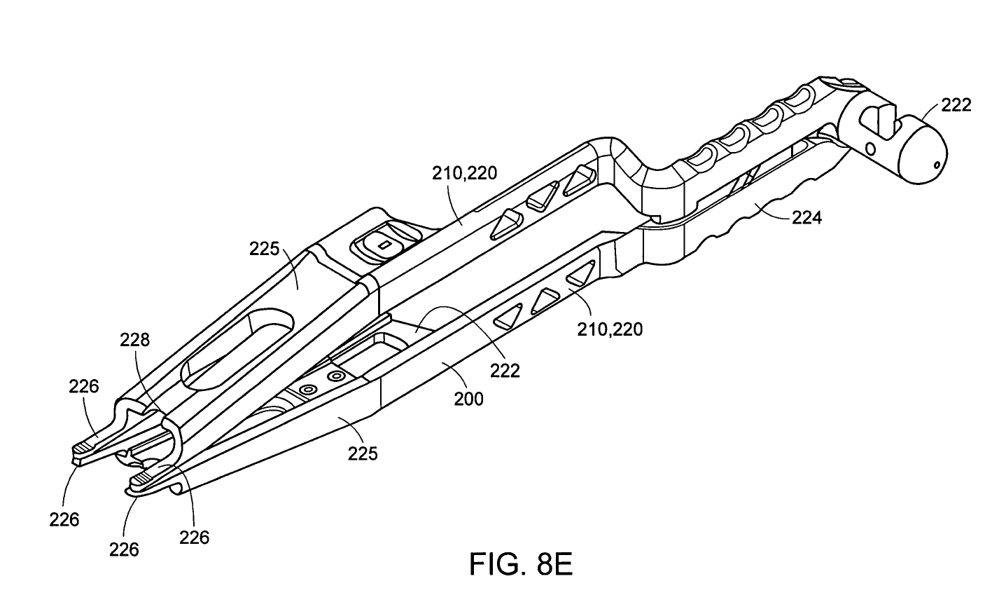

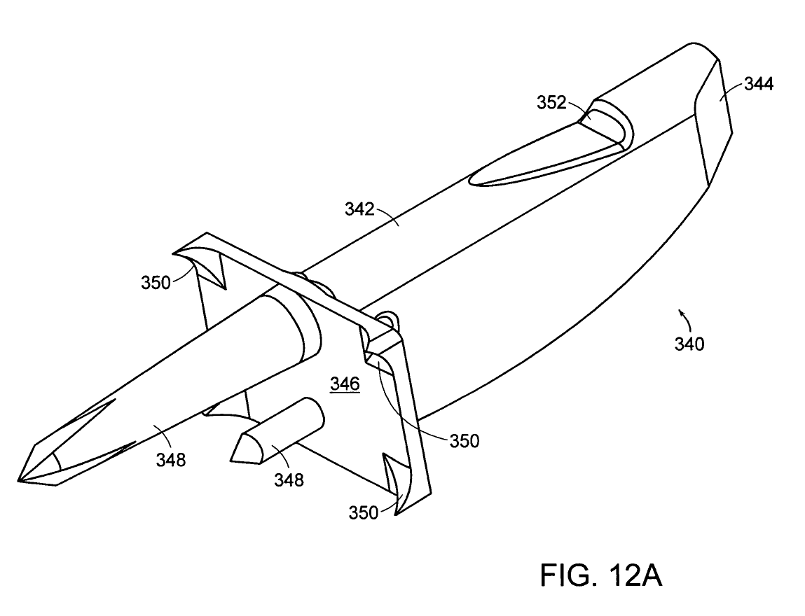

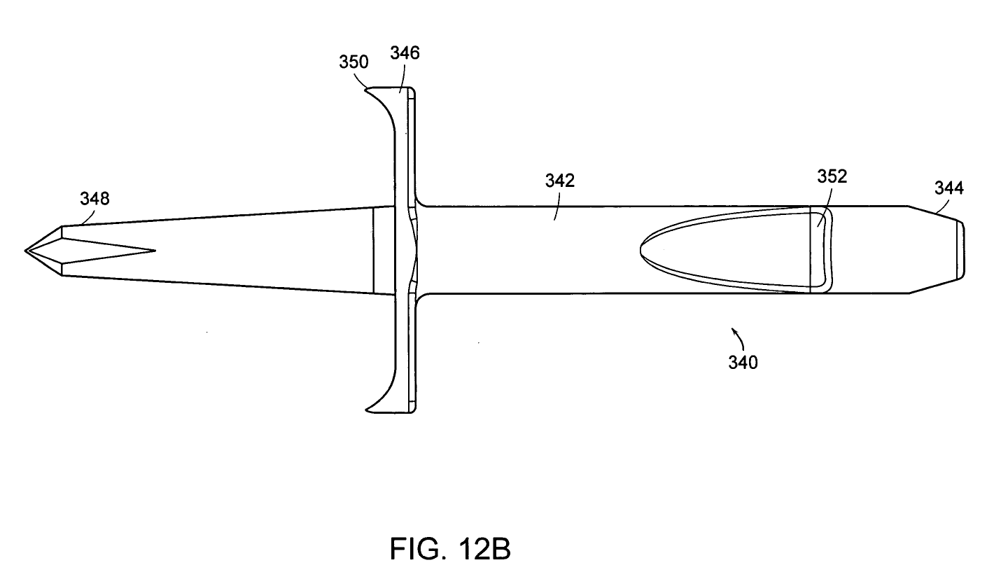

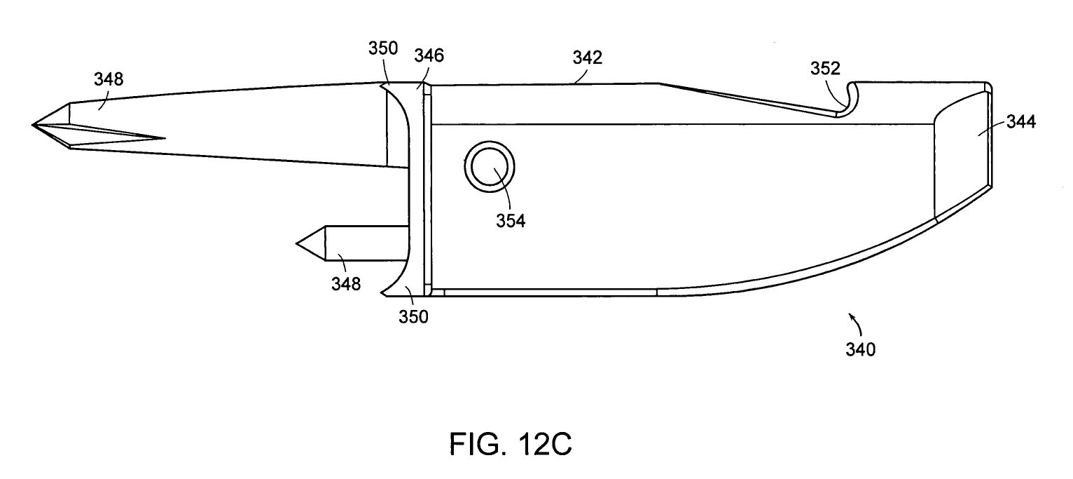

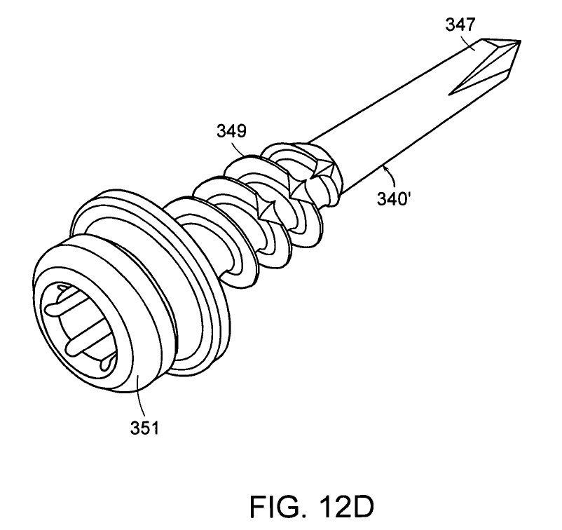

FIGS. 8A-8C show one embodiment of distraction instrument 200 according to the invention. FIGS. 8D and 8E show other embodiments of distraction instrument 200 of the invention. In general, distraction instrument 200 allows implants, trials, or instruments to be loaded in and out of distraction instrument 200 while maintaining correct alignment on midline marker 340 (FIGS. 12A-12C). Distraction instrument 200 includes diametrically opposing arms 210, 220, distraction mechanism 222 (FIGS. 8A-8C and 8D), and handle 224. Each arm 210, 220 includes insertion tip 226, midline marker slot 228, and guide face 232. Although guide face 232 is shown as having a smooth surface, it should be understood that guide face 232 can include a notch or a slot to allow implants, trials, or instruments to be loaded in and out of distraction instrument 200 as previously described above. In some embodiments, arms 210, 220 of distraction instrument 200 are spring 236 (FIGS. 8A-8C) loaded open in its normal position 205. In other embodiments, these arms are unbiased, so that they open and close simply by passing instruments or implants therethrough. As shown in FIG. 8E, distraction instrument 200 can include removable ends 225. Removable ends 225 can be selected based upon the amount of distraction and endplate angle needed.

Trial Insertion Instrument

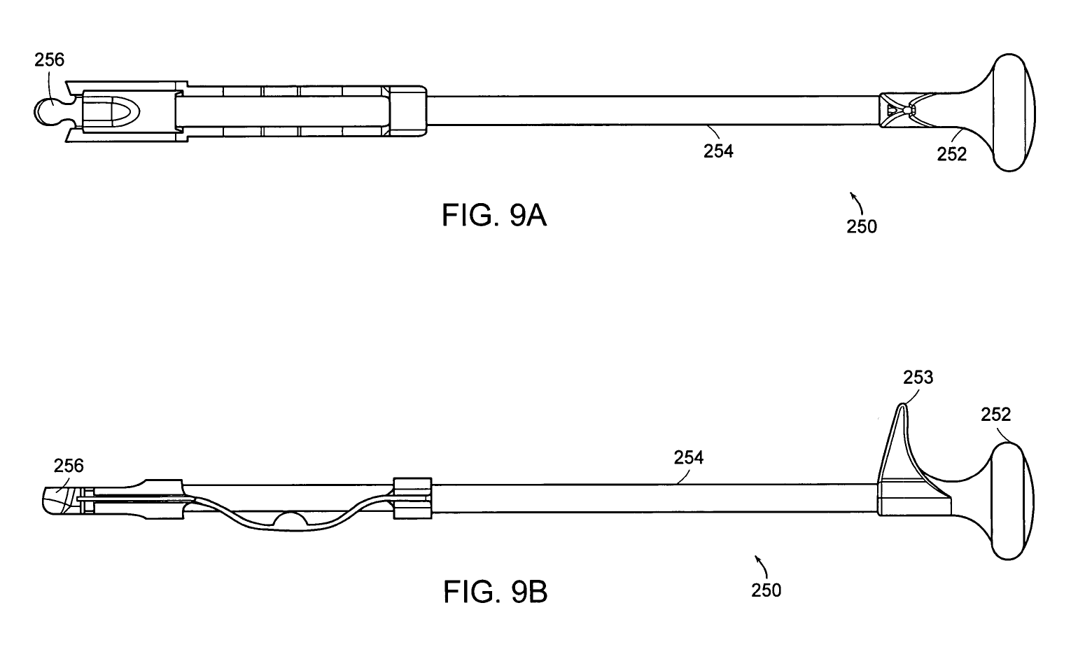

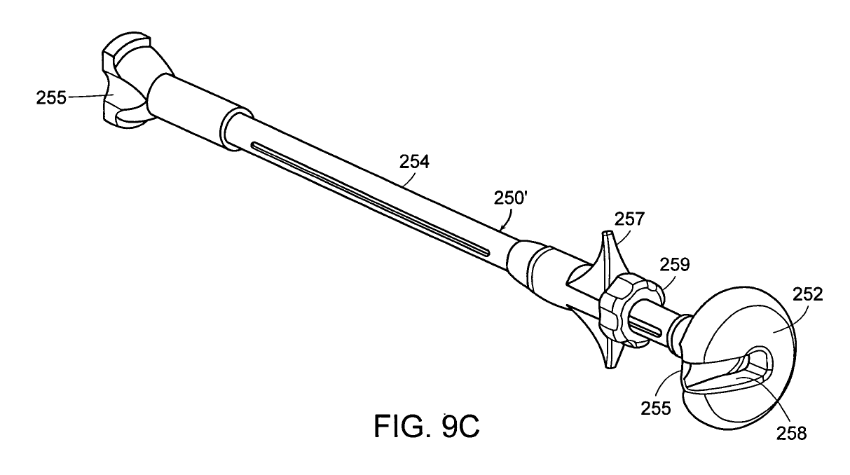

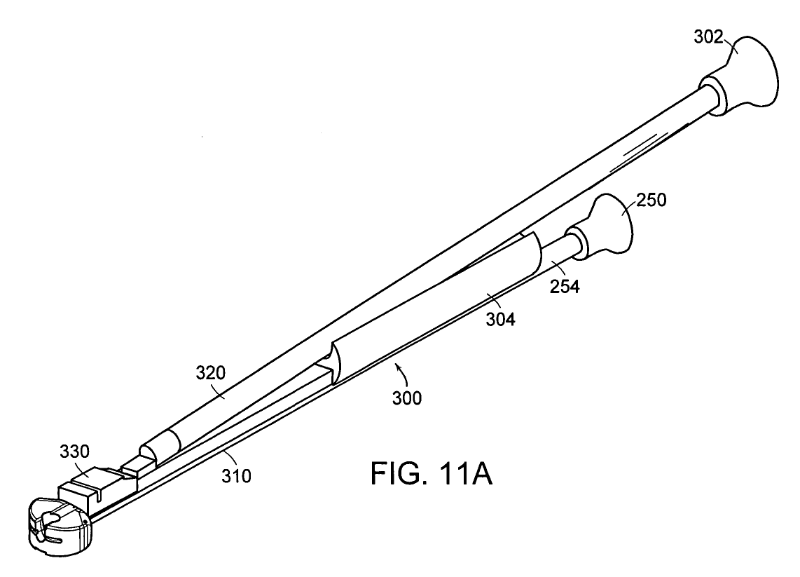

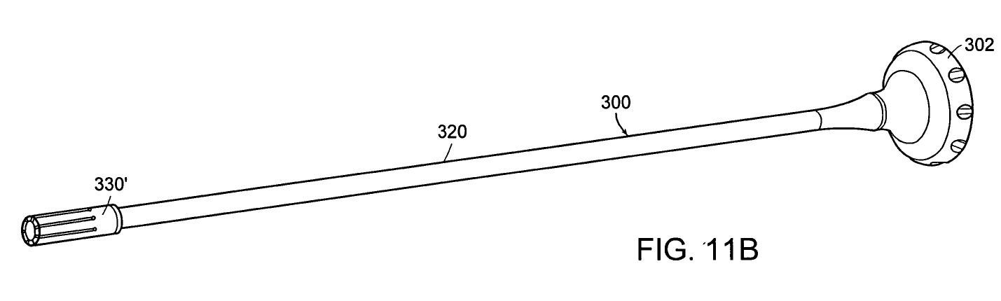

FIGS. 9A and 9B show trial insertion instrument 250. Trial insertion instrument 250 includes handle 252, shaft 254, and mateable head 256 for mating to trial spacer 260. Mateable head 256 can be made from a radiolucent material to allow for X-ray visualization of trial spacer 260. Pointer 253 provides a visual guide for determining the orientation of the trial spacer head within the disc space. In another embodiment, as shown in FIG. 9C, trial insertion instrument 250′ includes handle 252, shaft 254, grooves 255, release handle 257, and locking nut 259. Grooves 255 allow shaft 320 of midline marker insertion instrument 300 (FIGS. 11A and 11B) to be guided into the intervertebral disc space. Release handle 257 (FIG. 9C) allows trail spacer head 260′ (FIGS. 10D and 10E) to be removable coupled to trial insertion instrument 250′. Locking nut 259 (FIG. 9C) locks release handle 257 in a fixed position. This instrument also includes a slaphammer connection port 258 for easy removal.

Trial Spacer Head

FIGS. 10A-10D show trial spacer head 260. Trial spacer head 260 includes superior 264 and inferior 266 surfaces. Each surface 264, 266 includes at lease one groove 262 for slidably mating with arm 210/220 of distraction instrument 200 (e.g., FIG. 8D). FIGS. 10E and 10F show another embodiment of trial spacer head 260, denoted as 260′. Trial spacer head 260′ includes radiopaque pins 261 and mateable end 263. Mateable end 263 can be removable coupled to trial insertion instrument 250′ (FIG. 9C). Trial spacer head 260′ can contain a radiopaque agent for viewing via x-ray.

Midline Marker Insertion Instrument

FIG. 11A shows midline marker insertion instrument 300. Midline marker insertion instrument 300 facilitates placement of midline marker 340 (FIG. 12A). Midline marker insertion instrument 300 includes proximal end 302, distal tip 330, capturing device 304, spacing element 310, and insertion shaft 320. As explained above, midline marker insertion instrument 300 slides down shaft 254 of trial insertion instrument 250 having midline marker 340 (FIGS. 12A-12C) loaded into distal tip 330. Distal tip 330 is mated to the shaft by a hinge, and includes a dial to provide variable vertical placement of the midline marker 340. Capturing device 304 couples to shaft 254 of trial insertion instrument 250 to facilitate alignment and insertion of midline marker 340. Spacing element 310 can be used between insertion shaft 320 and shaft 254 of trial insertion instrument 250 to provide the correct height for inserting midline marker 340 into a face of a vertebra. FIG. 11B shows another embodiment of marker insertion instrument 300. Distal tip 330′ allows for insertion and retention of midline marker 340′ shown in FIG. 12D.

Midline Marker

FIGS. 12A-12C show midline marker 340. Midline marker 340 is an intra-operative marker that retains and communicates the ideal implant location throughout the entire implant procedure. Midline marker includes body element 342, tapered end 344 and attachment end 346. Attachment end 346 includes at least two pins 348 for insertion into a face of a vertebra as explained above. Pins 348 prevent midline marker 340 from rotating during the implant procedure. Attachment end 346 can include retention spikes 350 to further prevent rotation of midline marker 340. Body element 342 can include notch 352 and/or hole 354 to allow for removal of midline marker 340 once the implant procedure is completed. FIG. 12D shows another embodiment of midline marker 340, denoted as 340′. Midline marker 340′ includes insertion end 347, threaded mid-section 349, and head 351. Head 351 mates with distal tip 330′ of midline marker insertion instrument 300 (FIG. 11B).

Although FIGS.12A-c show the midline markers as being inserted into the bone, any method of fixing the position of the midline markers relative to a face of the bone is contemplated as within the scope of the invention. In some embodiments thereof, the midline markers are screwed into the bone. In others, the midline markers are clamped onto the bone. In others, the midline markers abut the face of the bone.

Endplate Shaping Instrument

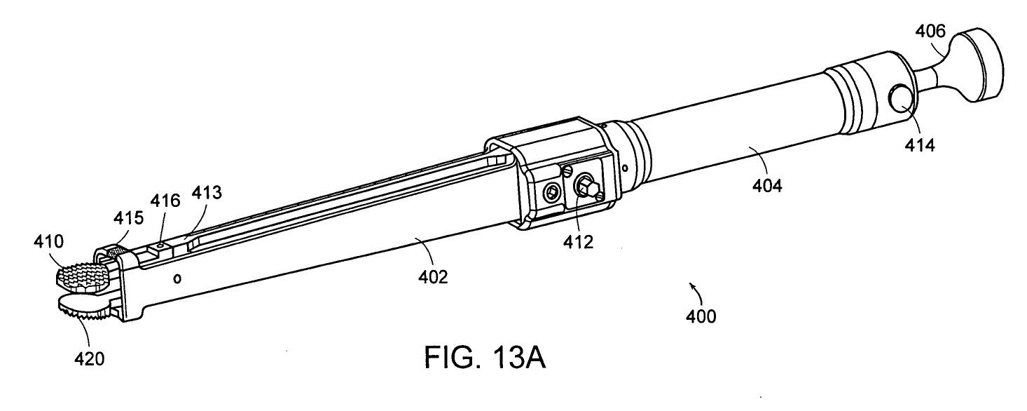

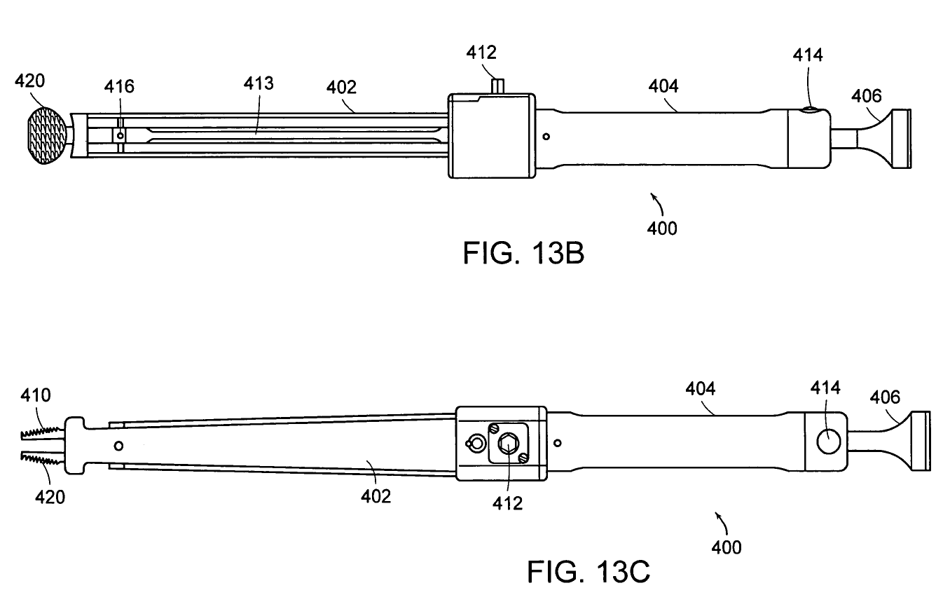

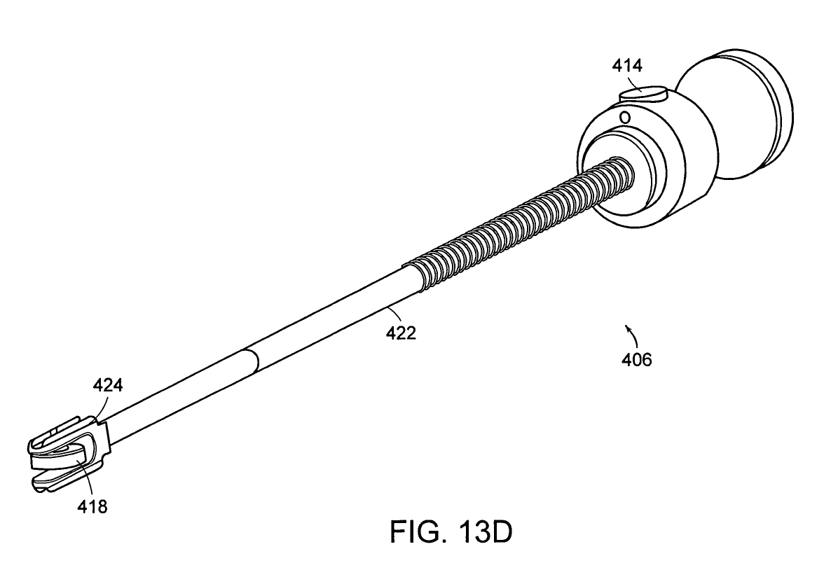

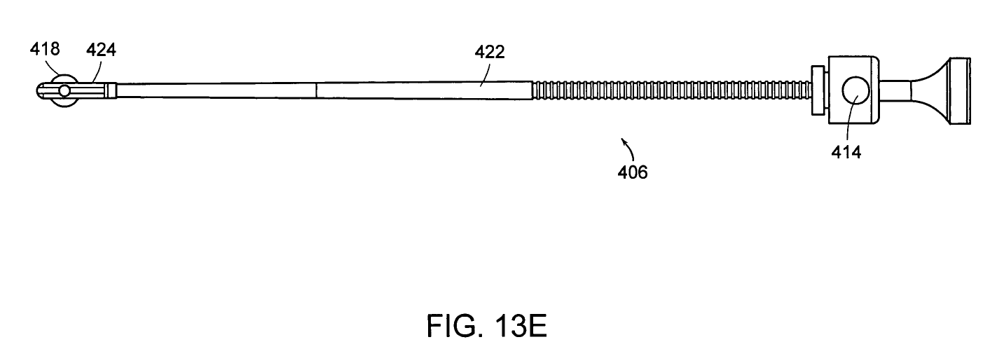

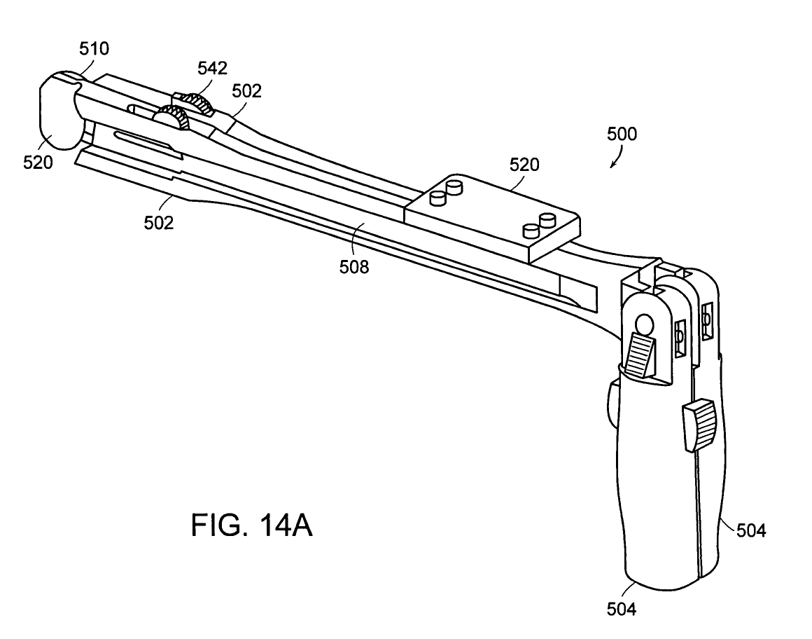

FIGS. 13A-13E show endplate shaping instrument 400 according to an embodiment of the invention. Endplate shaping instrument 400 includes frame 402, handle 404, spreader shaft 406, driving cam shaft 412, locking pushbutton 414, and cutter blades 410, 420. Centering slot 415 accepts midline marker 340 (FIGS. 12A-12C) to provide correct alignment when shaping boney vertebral bodies. Cutter blades 410, 420 can be adjusted by height (distance between the cutting surfaces of the cutters) and are inserted into the vertebrae in a collapsed state. Cutter blades 410, 420 can be spread apart to establish a proper tension for the cutting action. The cutting action of cutter blades 410, 420 is achieved by reciprocating cutters blades 410, 420 in an anterior-posterior (AP) direction. The energy for reciprocation is provided by a standard power tool (not shown) usually available in the operating room. The power tool is attached to driving cam shaft 412 and provides rotational motion that is converted into reciprocating movement of cutter blades 410, 420. Locking pushbutton 404 locks spreader shaft 406 in a fixed position. In combination with discrete graduations provided on the associated rod, locking pushbutton 404 also provides the ability to discretely adjust the height of cutter blades 410, 420. Spreader shaft 406 can include graduations or markings which provide the height of cutters 410, 420 to the operator.

The driving mechanism includes two cutting shafts 413 and a pivot block (not shown). Cutting shafts 413 are attached to the pivot block and rotate around their points of attachment. Driving cam shaft 412 is inserted into a slot in the pivot block and moves the pivot block up and down converting the rotational motion into reciprocating movement of cutting shafts 413. Cutting shafts 413 can be spread apart, but when the cutter blades 410, 420 are inserted into the intervertebral space, cutting shafts 413 are pressed against roller 418 (FIGS. 13D and 13E) of spreader shaft 406. Roller 418 spreads cutter blades 410, 420 apart such that cutter blades engage boney vertebral endplates. Roller 418 is interchangable depending upon the distance required. Cutting shafts 413 can be pressed together with torsion or compression springs for initial centering of the cutter blades 410, 420 for ease of insertion.

FIGS. 13D and 13E show spreader shaft 406. Spreader shaft 406 includes rod 422, fork 424, roller 418, and locking pushbutton assembly 414. There are different sizes (diameter) of roller 418 depending on the height of that needs to be achieved. Fork 424 includes slots on both sides that engaged rails located inside and along frame 402 which provide centering of roller 418, cutting shafts 413, and cutter blades 410, 420. Cutting shafts 413 get spread apart and cutter blades 410, 420 get adjusted to the required height when spreader shaft 406 is pushed down endplate shaping instrument 400.

Cutter blades 410, 420 include teeth with chip breakers on a side facing the endplate to be shaped. The direction of cutting is out of the intervertebral space only. The boney endplates get shaped to the shape of cutter blades 410, 420.

Endplate Insertion Instrument

FIGS. 14A-14D show an embodiment of endplate insertion instrument 500. Endplate insertion instrument 500 is used for initial delivery of the implant without the implants articulating core 620 (FIG. 6A). Endplate insertion instrument includes diametrically opposing arms 502, handles 504, and tines 540 (FIG. 14B). Each arm 502 includes a slot 508 for slidably mating with guide face 232 of distraction instrument 200 (FIGS. 8A). Each arm 502 also includes a channel 509 for slidably mating with fins 614 of the core insertion instrument 600 (FIGS. 6A). Mounting plate 521 couples opposing arms 502 and allows arms 502 to be opened or closed depending upon the procedure to be performed. Tines 540 hold endplates 510, 520 to arms 502 until released by distraction. Endplates 510, 520 can be any angle or size as well as mismatched superior and inferior. An interchangeable insertion stop 530 can be used to establish endplate insertion depth. Interchangeable insertion stop 530 can be chosen from a kit of interchangable insertion stops 530 to match the chosen trial spacer 260. Pushbutton 542 allows for the anterior-posterior adjustment of insertion stop 530. FIG. 14E shows another embodiment of endplate insertion instrument 500, denoted as 500′. Endplate insertion instrument 500′ is essentially the same as endplate insertion instrument 500 except channel 509 (FIG. 14B) has been replaced by guide 505. Also removable end 503 has been included to interchange tines 540 depending upon the size of the endplate.

Now referring to FIG. 14E, in one embodiment, hinge 521 has a torsion spring to bias the handles apart. When the handles are in their closed position, the endplates held by the instrument can not shift along the anterior-posterior axis. However, if the handles are in their open position, independent adjustment of the endplates is possible.

Alignment tabs 551,552 maintain the medial-lateral alignment of the endplates during their insertion. In other embodiments, a pin-and-slot alignment mechanism may be used.

Core Trial Instrument

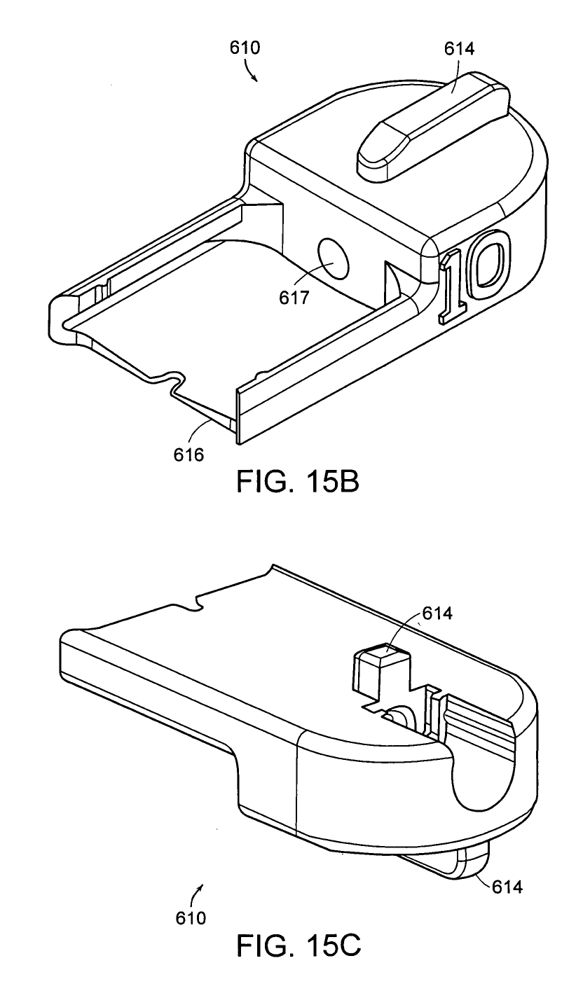

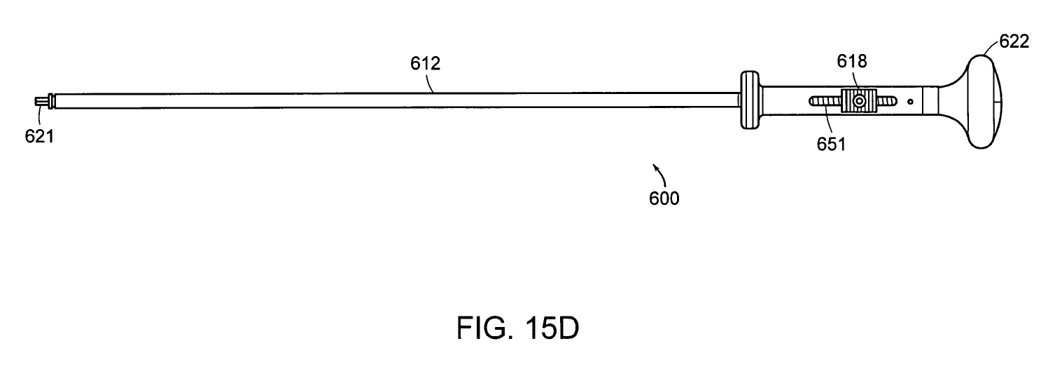

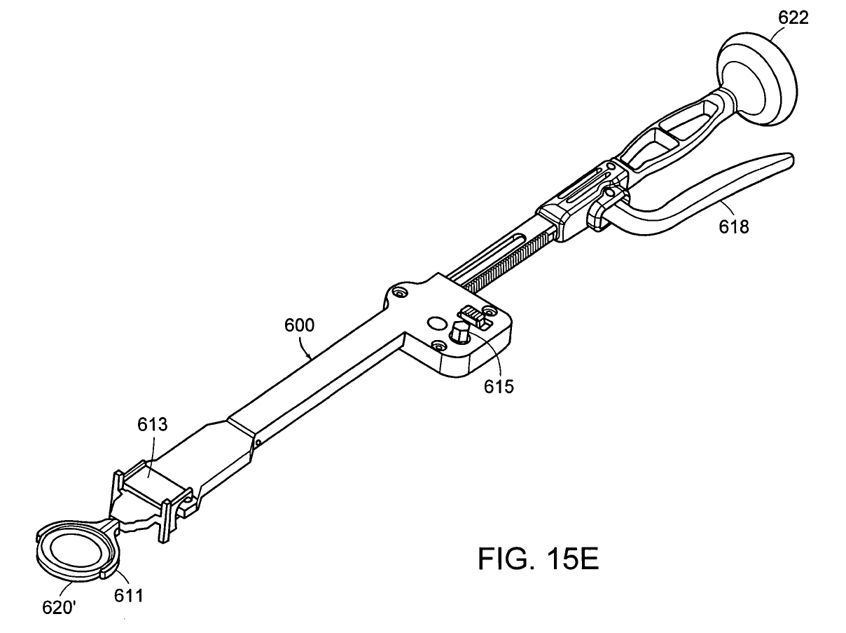

There are three pieces of information the surgeon should know when selecting an appropriately sized implant. These are a) footprint or size of the implant, b) lordotic angle, and c) core height. Whereas the footprint and lordotic angle are determined during the trialing process, core height is determined with the core trialing instrument. FIGS. 20,21 illustrate two embodiments of this core trial instrument and both are used in a similar manner with their corresponding endplate insertion instruments. Both of these core trial instruments comprise modular ends 900,900′, the heights of which correspond to the core heights, a shaft 902,902′, and a handle 904,904′. Additionally, the modular ends both contain surfaces that keep the instrument centered as it is passed down the endplate insertion instrument. It should be noted that the modular end 900′ used with the instrument shown in FIG. 21 is identical to the distraction block (613) shown with the core insertion instrument in FIG. 15E above. Preferably, the instrument kits contains a modular end corresponding to each core height. Therefore, the surgeon can advantageously pass this core trailing instrument down the endplate insertion instrument and evaluate the height via x-ray. If the evaluated height is determined to be not optimal, the instrument will be removed and the modular end will be replaced with a different size. The process can then be repeated until the correct height has been determined. When this information is obtained, the corresponding core height can be selected.

Core Insertion Instrument

FIGS. 15A-15D show core insertion instrument 600. Core insertion instrument 600 is used following the successful placement of the endplates 510, 520. Core insertion instrument 600 includes removable cassette 610, insertion shaft 612, core insertion knob/handle 618, pushrod 621, and handle 622. Removable cassette 610 includes fins 614 for slidably mating with channels 509 within endplate insertion instrument 500 to maintain correct alignment while inserting core 620 between endplates 510, 520. Removable cassette 610 also includes push rod hole 617 which allows pushrod 621 to move core 620 from removable cassette 610. Removable cassette 610 can be chosen from a kit of cassettes to match the height of core 620. In other embodiments, the cassette may be made of a disposable plastic and packaged with the core. Pushrod 621 is slidably disposed within insertion shaft 612 and is operable via insertion knob/handle 618. Spring 651 maintains pushrod 621 with insertion shaft 612 until insertion knob 618 is moved toward core 620. FIG. 15E shows another embodiment of core insertion instrument 600, denoted as 600′. Core insertion instrument 600′ is essentially the same as core insertion instrument 600 except cassette 610 has been replaced by claw 611. Claw 611 attaches to core 620′ by compressing core 620′. Core insertion instrument 600′ also includes distraction block 613 and ratchet mechanism 615. Distraction block 613 slidably engages guide 505 of endplate insertion instrument 500′ to distract the intervertebral space. Ratchet mechanism 615 is used to withdraw block 613, thereby reducing the intervertebral space and collapsing the endplates onto the core. Handle 618 is then squeezed and the instrument is removed, leaving the core in place.

Retention Clip Insertion Instrument

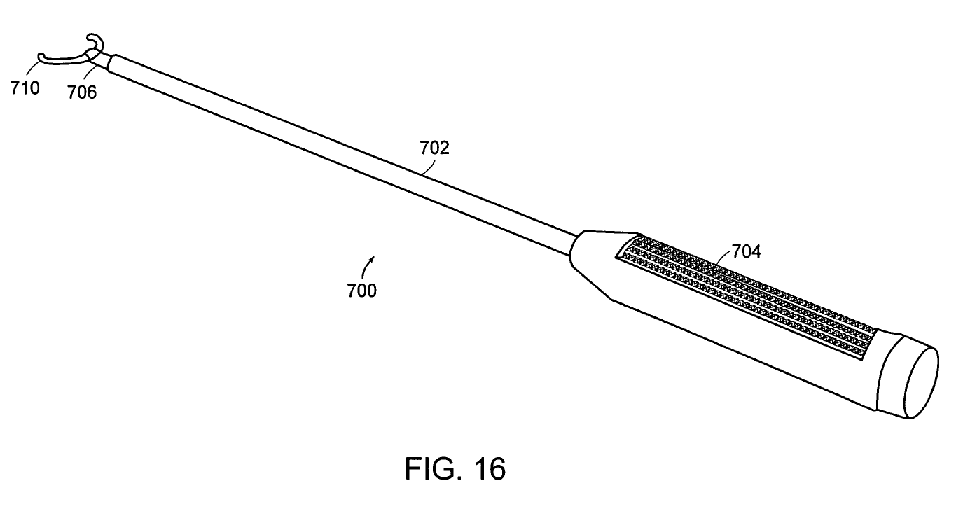

FIG. 16 shows retention clip insertion instrument 700. Retention clip insertion instrument 700 includes shaft 702, handle 704, and attachment point 706. Attachment point 706 “grips” onto a hole and beveled edge located on the anterior aspect of retention clip 710. After the artificial disc has been successfully implanted, retention clip 710 is fixed about internal rails on an inferior endplate of the artificial disc to permanently retain core 620. Once clip 710 is affixed to the internal rails retention clip insertion instrument 700 is removed.

Retention Clip Removal Instrument

FIG. 17 shows retention clip removal instrument 800. Retention clip removal instrument 800 includes two handles 802 movably attached at pivot point 804. In some embodiments having a longer length, multiple hinges and/or linkages may be used between the handles and pivot points 804. Retention clip removal instrument 800 is an extraction tool which is used in the event the core 620 needs to be changed (to modify the disk height), or if the implant needs to be removed. Retention clip removal instrument 800 distorts and retains retention clip 710 for its disposal, allowing core 620 to slide anteriorly from the artificial disc

Verification Instrument

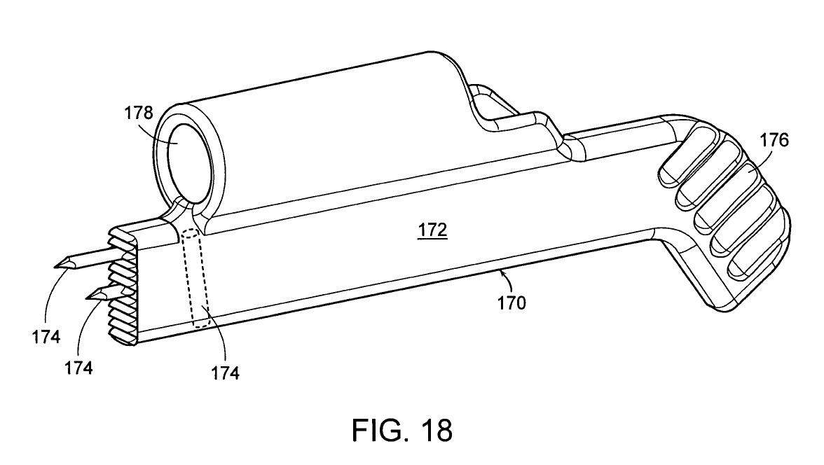

FIG. 18 shows verification instrument 170. Verification instrument 170 includes radiolucent body 172, radiopaque pins 174, handle 176, and midline marker guide 178.

Core insertion with the instruments shown in FIGS. 15A and 15E has been previously discussed. In that method, the core insertion instrument is passed down the endplate insertion instrument, and, in the process of doing so, distracts the disc space.

In some embodiments, there is provided an alternate method for placing the implant endplates and core. This methodutilizes the essentially identical trialing and midline marking methods as discussed above but with different instrumentation associated with placing the endplates, distracting the disc space, and placing the core.

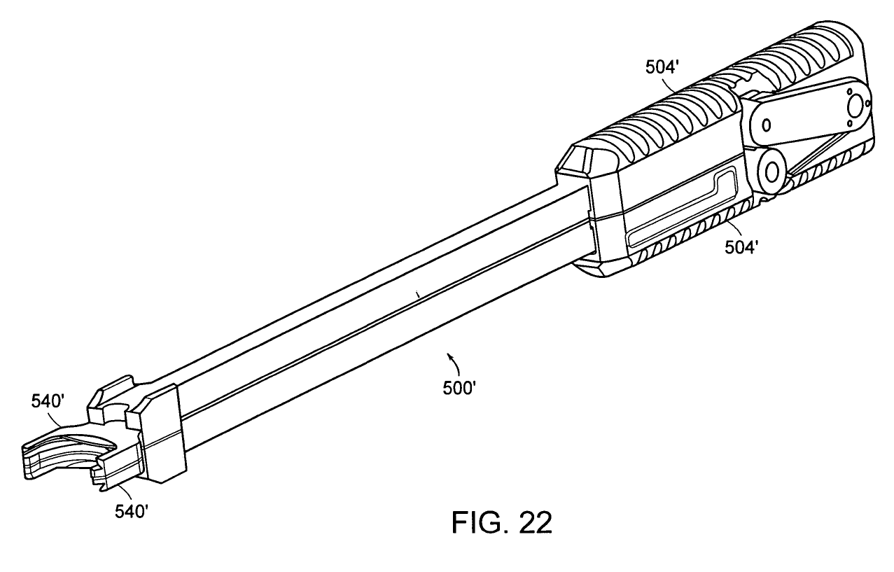

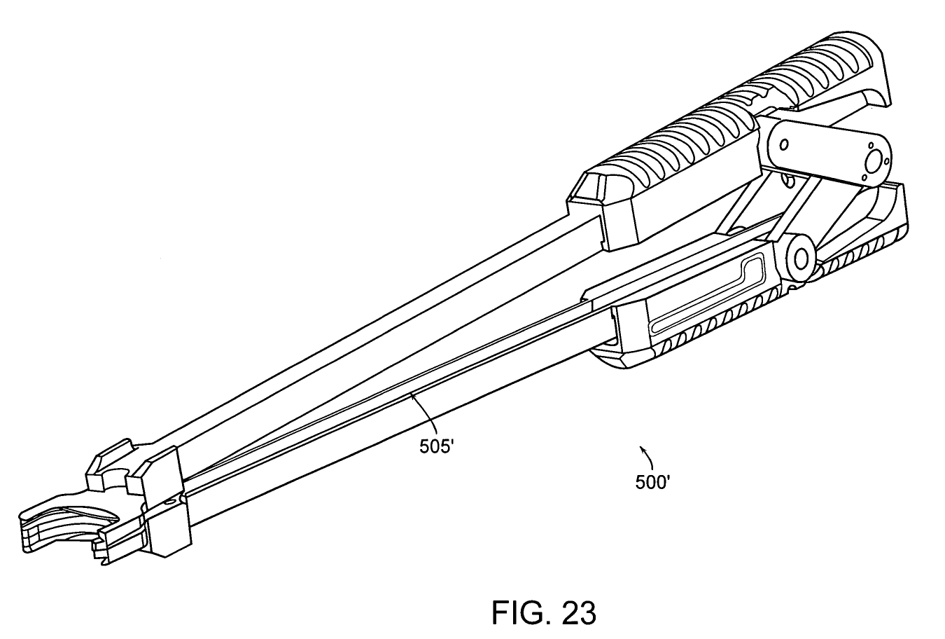

Now referring to FIGS. 22 and 23, in this alternate embodiment, the endplate insertion instrument 500′ holds the implant in an identical manner as the 500′ endplate insertion instrument (540′) shown in FIG. 14E. In FIG. 22, the instrument 500′ is shown in the closed position. In this configuration, the instrument 500′ is passed through the distraction instrument 200 (as in FIG. 8E). Once the endplate insertion instrument 500′ has reached its final placement, the distraction instrument 200 is removed and the endplate insertion instrument 500′ is allowed to open (as shown in FIG. 23), thereby engaging the endplates and permitting the passage of the core trialing and core insertion instruments.

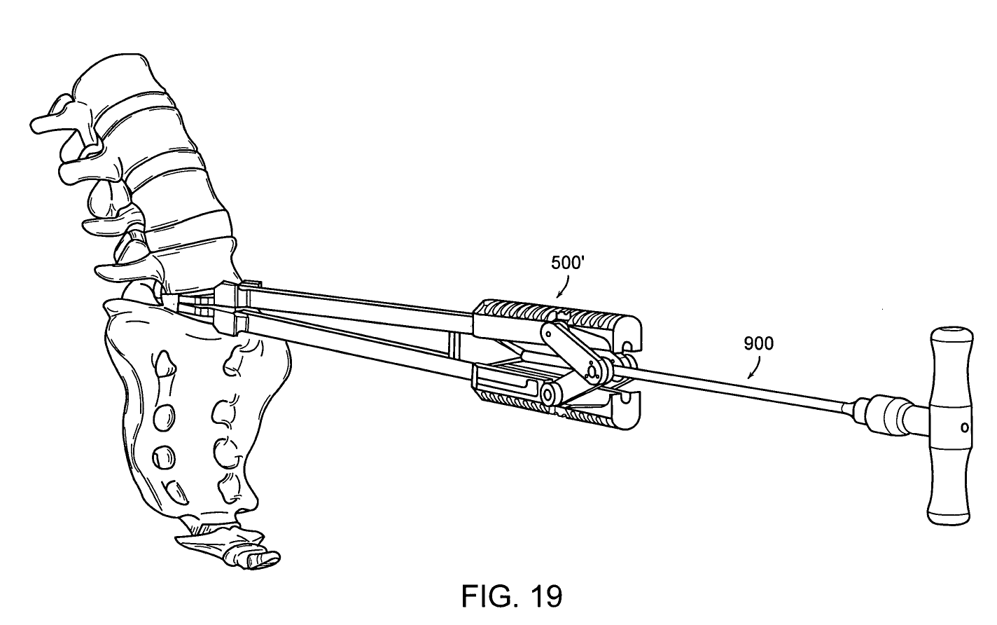

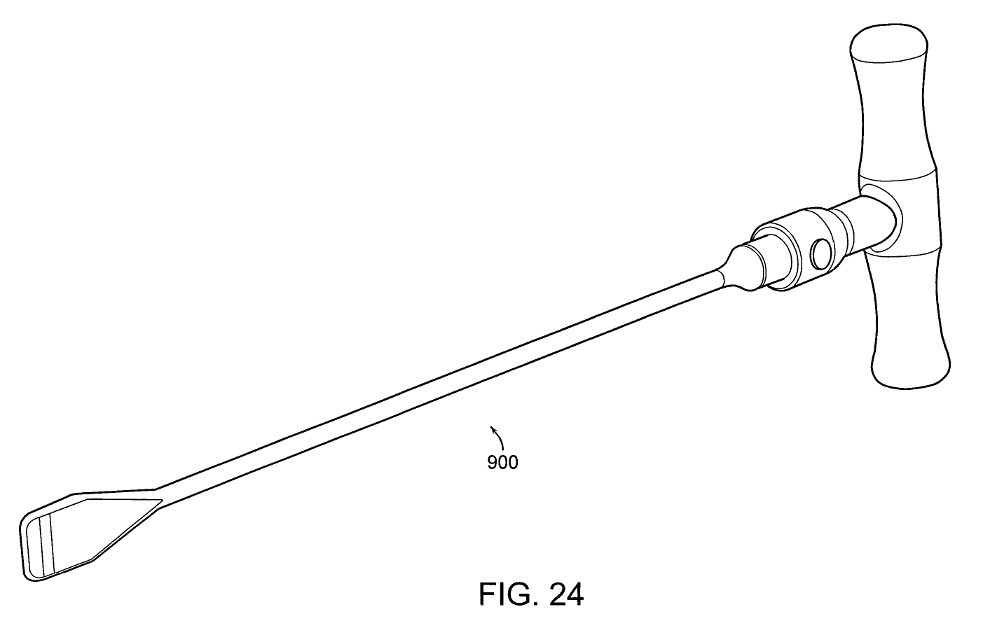

This alternative method separates the acts of distracting the disc space and core placement. Now referring to FIG. 24, a spreader 900 is passed down the endplate insertion instrument 500′ shown in FIG. 19. Since the instrument kit preferably contains one spreader height for each core height, core trialing is preferably conducted with this spreader instrument. Once the appropriate core height has been determined, the spreader is left in place, and the core is placed with a core insertion instrument such as the core insertion instrument catalog No. 2869-22-000 manufactured by DePuy Spine of Raynham, Mass. (currently the same instrument used in the CentreLigne Set to place the core)(where is this?) off the primary axis of the endplate insertion instrument.

Equivalents

While this invention has been particularly shown and described with references to preferred embodiments thereof, it will be understood by those skilled in the art that various changes in form and details may be made therein without departing from the scope of the invention encompassed by the appended claims.

1. An anterior method for implanting an artificial disc in an intervertebral space of the human body, comprising:
fixing a position of a midline marker relative to a face of a vertebral body for instrument alignment and artificial disc placement.

2. The anterior method of claim 1, further comprising verifying a disc for artificial disc implantation.

3. The anterior method of claim 2, wherein verifying a disc for artificial disc implantation includes:
centering a verification instrument on the disc;
inserting at least one radiopaque pin extending from the verification instrument into the disc;
visualizing via X-ray the radiopaque pin in the disc; and
removing the verification instrument from the disc after visualization.

4. The anterior method of claim 3, further comprises:
inserting the midline marker in a guide of the verification instrument; and
impacting a proximal end of the midline marker until the midline marker is embedded in the face of the vertebral body.

5. The anterior method of claim 1, further comprises:
preparing the disc for artificial disc implantation; and
selecting an artificial disc for implantation.

6. The anterior method of claim 5, wherein preparing the disc for artificial disc implantation includes:
removing a window from the annulus of the disc, where the window is the width of an artificial disc implant; and
removing the nucleus pulposus of the disc.

7. The anterior method of claim 5, wherein selecting an artificial disc for implantation, includes:
distracting the intervertebral space with a distraction instrument;
inserting at least one trial spacer into the distracted intervertebral space with a trial spacer insertion instrument, the trial spacer instrument guided into the intervertebral spacer by the distraction instrument; and
removing the trial spacer insertion instrument from the intervertebral space.

8. The anterior method of claim 7, further comprises:
contacting the trial spacer insertion instrument with the pin insertion instrument;
inserting the midline marker in the face of the vertebral body with the pin insertion instrument while being guided by the trial spacer insertion instrument; and
removing the pin insertion instrument from the midline marker.

9. The anterior method of claim 5, further comprises shaping adjacent endplates of the vertebral bodies which define the intervertebral space with an endplate shaping instrument being guided by the midline marker.

10. The anterior method of claim 9, wherein the step of shaping adjacent endplates of the vertebral bodies, comprises:
aligning an endplate shaping instrument with the midline marker;
inserting shaping blades of the endplate shaping instrument into the intervertebral space; and
shaping the adjacent endplates of the vertebral bodies with the shaping blades.

11. The anterior method of claim 5, further comprising:
implanting the artificial disc in the intervertebral space using the midline marker as a guide;
removing the midline marker; and
closing the surgical site.

12. The anterior method of claim 11, wherein implanting the artificial disc in the intervertebral space includes:
inserting a distraction instrument into the intervertebral space using the midline marker as a guide;
inserting endplates of the artificial disc into the intervertebral space using an endplate insertion instrument, the endplate insertion instrument guided by the distraction instrument;
removing the distraction instrument from the intervertebral space, thereby allowing the endplates of the artificial disc to engage vertebral endplates;
inserting a core between the endplates of the artificial disc using a core insertion instrument, the core insertion instrument guided by the endplate insertion instrument;
removing the core insertion instrument from the endplate insertion instrument; and
removing the endplate insertion instrument from the intervertebral space.

13. The method of claim 12, wherein inserting a core between the endplates further includes securing the core between the endplates of the artificial disc with a retention clip.

14. A kit for implanting an artificial disc in an intervertebral space of the human body, comprising:
artificial disc insertion instruments for implanting the artificial disc into the prepared intervertebral space; and
a midline marker for guiding the artificial disc insertion instruments into the prepared intervertebral space.

15. The kit of claim 14, wherein the site preparation instruments include a verification instrument, the verification instrument comprising:
a radiolucent body, the body having a proximal end and a distal end;
a handle at the distal end of the body; and
at least one radiopaque pin at the proximal end of the body.

16. The kit of claim 15, wherein the verification instrument further comprises a guide on a surface on the body for mating with a midline marker insertion instrument.

17. The kit of claim 14, wherein the artificial disc insertion instruments include:
a distraction instrument for distracting the intervertebral space;
a trial spacer insertion instrument and various trial spacer heads for inserting into the distracted intervertebral space;
an endplate insertion instrument for inserting endplates of the artificial disc into the distracted intervertebral space; and
a core insertion instrument for inserting a core between the endplates of the artificial disc.

18. The kit of claim 17, wherein the distraction instrument includes:
a body element;
superior and inferior arms coupled to the body; and
a centering feature in at least one arm adapted to align with a midline marker.

19. The kit of claim 17, wherein the various trial spacer heads include:
a radiolucent body having superior and inferior surfaces;
diametrically opposing grooves on the superior and inferior surfaces, each groove for maintaining a centered position on superior and inferior arms of the distraction instrument; and
radiopaque pins within the radiolucent body for X-ray visualization.

20. The kit of claim 17, wherein the endplate insertion instrument includes:
a body element; and
a pair of diametrically opposing arms coupled to the body, each arm having guides for mating with the distraction instrument and the core insertion instrument.

21. The kit of claim 17, wherein the artificial disc insertion instruments further include a core trial instrument.

22. The kit of claim 17, wherein the core insertion instrument includes:
a body element; and
guides on superior and inferior surfaces of the body, each guide for maintaining a centered position on superior and inferior arms of the endplate insertion instrument.

23. The kit of claim 17, further including:
an endplate shaping instrument, the endplate shaping instrument having at least one centering feature for capturing the midline marker.

24. The kit of claim 17, further including:
a retention clip insertion instrument for securing the core to an endplate of the artificial disc; and
a retention clip removal instrument for removing the core form the endplate of the artificial disc.

25. A verification instrument for determining a disc for artificial disc replacement, comprising:
a radiolucent body, the body having a proximal end and a distal end;
a handle at the distal end of the body; and
at least one radiopaque pin at the proximal end of the body.

26. The verification instrument of claim 25, wherein the at least oneradiopaque pin includes a first portion extending horizontally from the proximal end of the body and a second portion extending vertically within the proximal end of the body.

27. The verification instrument of claim 25, wherein the verification instrument further comprises a guide on a surface of the radiolucent body.

28. The verification instrument of claim 27, wherein the guide is selected from the group consisting of a slot and a bore.

29. A midline marker for providing instrument alignment and artificial disc placement, comprising:
a body element, the body element having a proximal end and a distal end; and
at least two protrusions, parallel to each other and forming the distal end of the body element.

30. The midline marker of claim 29, further including retention spikes extending from the attachment end of the body element.

31. An endplate shaping device, comprising:
a frame, the frame having a proximal end and a distal end;
a handle coupled to the proximal end of the frame;
a driving mechanism, the driving mechanism disposed within the frame;
two cutting shafts, each cutting shaft having a proximal end and a distal end, the proximal end of each shaft separately coupled to a pivot block on the driving mechanism and rotatable around their points of attachment, and the distal end of each cutting shaft extending from the distal end of the frame; and
a pair of cutter blades, each cutter blade coupled to a respective distal end of each cutting shaft.

32. The endplate shaping device of claim 31, further comprising a spreader shaft slidably disposed between the two cutting shafts and extending from the proximal end of the frame.

33. The endplate shaping device of claim 32, wherein the spreader shaft includes:
a rod having a proximal end and a distal end;
a fork element coupled to the distal end of the rod;
a roller assembly coupled between the fork element; and
a locking pushbutton for locking of the rod within the frame.

34. The endplate shaping device of claim 33, further including discrete graduations on the rod to determine the distance between the cutter blades.

35. The endplate shaping device of claim 31, further comprising at least one centering feature located on a distal end of the frame for mating with the midline marker.

36. A distraction instrument, comprising:
a body element;
a pair of diametrically opposing arms coupled to the body, at least one arm including a centering feature adapted to align with a midline marker; and
a distraction mechanism movably coupled between the diametrically opposing arms.

37. The distraction instrument of claim 36, wherein the pair of diametrically opposing arms are removably coupled to the body.

38. An endplate insertion instrument, comprising:
a body element;
a pair of diametrically opposing arms coupled to the body, the arms having first and second opposed surfaces respectively having first and second opposed alignment surfaces;
an endplate holder coupled to one end of each arm;
a handle portion coupled to an opposite end of the arm; and
a mounting plate, each arm slidably coupled to opposite ends of the mounting plate.

39. The endplate insertion instrument of claim 38, wherein the endplate holder includes an alignment feature.

40. The endplate insertion instrument of claim 38, wherein the endplate holder is removably coupled to the arm.

41. A core insertion instrument, comprising:
a body element, the body having a handle end and an insertion end; and
a pair of diametrically opposing guides on opposing surfaces of the insertion end.

42. The core insertion instrument of claim 41, wherein the insertion end is removably coupled to the body.

43. The core insertion instrument of claim 41, wherein the guides are removably coupled to the insertion end.

44. A trial spacer head for determining a correct-sized artificial disc, comprising:
a body element having superior and inferior surfaces;
diametrically opposing grooves on the superior and inferior surfaces of the body; and
radiopaque pins within the radiolucent body for X-ray visualization.

45. The trial spacer head of claim 44, wherein the body element comprises a radiopaque agent.

46. The trial spacer head of claim 44, wherein the radiopaque pins include two pairs of diametrically opposed radiopaque pins for determining alignment via X-ray.

47. The trial spacer head of claim 44, wherein one radiopaque pin of the pair of radiopaque pins has a greater diameter than the other radiopaque pin.

48. The trial spacer head of claim 44, wherein one radiopaque pin of the pair of radiopaque pins has a greater length than the other radiopaque pin.

49. A trial spacer head for determining a correct size of an artificial disc, the head comprising a composite comprising a radiolucent material and a radiopaque material.

50. The head of claim 49 wherein the radiolucent material is a polymer.

51. The head of claim 49 wherein the radiopaque material is barium sulfate.

52. A midline marker for providing instrument alignment and artificial disc placement, comprising:
a body element having a proximal end and a distal end,
at least one protrusion forming the distal end of the body element, and
a mating feature provided on the proximal end for mating with an insertion instrument.

53. The marker of claim 52 comprising a single protrusion.
